SEQUENCE LISTING (19 sequences)

SEQ ID NO: 1 - DNA, 55 bp, synthetic construct
  source 1..55: mol_type = other DNA
tcgtcggcagcgtcagatgtgtataagagacagctgctgaaccgctcttccgatc

SEQ ID NO: 2 - DNA, 60 bp, synthetic construct
  source 1..60: mol_type = other DNA
tcgtcggcagcgtcagatgtgtataagagacagtaagactgctgaaccgctcttccgatc

SEQ ID NO: 3 - DNA, 72 bp, synthetic construct
  source 1..72: mol_type = other DNA
tcgtcggcagcgtcagatgtgtataagagacaggctagacctaatgtgatctgctgaaccgctcttccgatc

SEQ ID NO: 4 - DNA, 63 bp, synthetic construct
  source 1..63: mol_type = other DNA
gtctcgtgggctcggagatgtgtataagagacagtctgcgttctgatttaatctgtatcaggc

SEQ ID NO: 5 - DNA, 22 bp, synthetic construct
  source 1..22: mol_type = other DNA
cagtgatagagatctccctatc

SEQ ID NO: 6 - DNA, 20 bp, synthetic construct
  source 1..20: mol_type = other DNA
gagatagatgcgggcttcca

SEQ ID NO: 7 - DNA, 24 bp, synthetic construct
  source 1..24: mol_type = other DNA
caccgggcacgggcagcttgccgg

SEQ ID NO: 8 - DNA, 24 bp, synthetic construct
  source 1..24: mol_type = other DNA
gaacccggcaagctgcccgtgccc

SEQ ID NO: 9 - DNA, 28 bp, synthetic construct
  source 1..28: mol_type = other DNA
caccgcccgaaggctacgtccaggagcg

SEQ ID NO: 10 - DNA, 28 bp, synthetic construct
  source 1..28: mol_type = other DNA
gaaccgctcctggacgtagccttcgggc

SEQ ID NO: 11 - DNA, 29 bp, synthetic construct
  source 1..29: mol_type = other DNA
caccgcagccaggtagtactgtgggtact

SEQ ID NO: 12 - DNA, 29 bp, synthetic construct
  source 1..29: mol_type = other DNA
gaacagtacccacagtactacctggctgc

SEQ ID NO: 13 - DNA, 31 bp, synthetic construct
  source 1..31: mol_type = other DNA
attaggatccagagtcatccagctggagccc

SEQ ID NO: 14 - DNA, 32 bp, synthetic construct
  source 1..32: mol_type = other DNA
taatctcgagtggggtttttcccattcccagg

SEQ ID NO: 15 - DNA, 32 bp, synthetic construct
  source 1..32: mol_type = other DNA
gtgtggtacgcagccacttcgagtacccacag

SEQ ID NO: 16 - DNA, 32 bp, synthetic construct
  source 1..32: mol_type = other DNA
ctgtgggtactcgaagtggctgcgtaccacac

SEQ ID NO: 17 - protein, 1002 aa, synthetic construct
MKMQDSVSKMKYRLGIDLGTTSLGWAMLRLDEQNEPYAVIRAGVRIFNNGRDPKTEASLAVARRLARQQRRTRDRKIRRKERLIGELVDMGFFPKDPVKRRQLASLDPFKLRTEALDRALSPEEFARAIFHLARRRGFKSNRKTDSGDTESSKMKEAIKRTLNELQNKGFRTVGEWLNMRHQQRLGTRSRIKNVPTGSGKQTTAYDFYLNRFMIEYEFDRIWEKQSQMNPGLFTNERKAILKDIIFYQRPLRPVEPGRCTFMPDNPRAPLALPQQQDFRIYQEVNNLRKIDPTSLLEVNLTLPERDRIVELLQRKPALTFDAVRKALCFNGTFNLEGENRSELKGNLTNCALAKKKLFGESWYSFDAHKRFEIVEHLLQEESEENLVSWLQKECNLSEEYAKNVASVRLPAGYGALCQEALDLILPYLKAEVITYDKAVQKAGMNHSELTLAQETGEILPELPYYGQYLKRHVGFGTGKPEDSAEKRYGKIPNPTVHIALNQLRTVVNALIRRYGKPTQIVIELARELKQNKKAKDQYRIEMNHNQNRNERIRADISMILGINPENVKRKDIEKQILWEELNLKDATARCCPYSGKQISAEMLFTDEVEIDHILPFSRTLDDSKNNKVVCIREANRIKGNRTPWEARKDFEKRGWSVEAMTARAQAMPKAKRFRFAEDGYKVWLKDFDGFEARALTDTQYMSRVAREYLQLICPGQTWSVPGQLTGMLRRFLGLNDILGVNGEKNRDDHRHHAVDACVIALTDRSMLQRISTASARAENKHLTRLLESFPAPWATFYEHVTRAVKSICVSHKPEHAYQGAMNEQTAYGLRPDGYVKYRQNGKVEHKKLNVIPQVSVKGTWRHGLNSDGSLKAYKGLKGGSNFCIEIVMGEGGRWEGDVITTYEAYQIVRAKGEAALYGSVSRSGKPLVMRLMQKDIVEMTLADGRCKMLLYIITQNKQMFFYRIENAGGGREDVSKRPGSLQKALAKKIIVSPIGDFRKEKL

SEQ ID NO: 18 - DNA, 24 bp, synthetic construct
  source 1..24: mol_type = other DNA
catgctattagtcagtgctataag

SEQ ID NO: 19 - RNA, 120 nt, synthetic construct
  source 1..120: mol_type = other RNA
gttctggtctaagttcatttcctaactgagaaatcagttaggaaatgggctttctccactaacaagctgagagatgcacaagatgcggggtcgctatatgcgaccattattcgtatcaaa

(19) United States
(12) Patent Application Publication
(10) Pub. No.: US 2026/0253672 A1
(43) Pub. Date: Aug. 27, 2026

(54) METHODS AND SYSTEMS FOR CRISPR-CAS PROTEIN PAM IDENTIFICATION

(71) Applicant: Alia Therapeutics Srl, Milano (IT)

(72) Inventors: Anna CERESETO, Trento (IT); Nicola SEGATA, Trento (IT); Matteo CICIANI, Schio (IT); Antonio CASINI, Trento (IT)

(73) Assignee: Alia Therapeutics Srl, Milano (IT)

(21) Appl. No.: 18/878,243

(22) PCT Filed: Nov. 18, 2022
(86) PCT No.: PCT/EP2022/082427

§ 371 Date: Dec. 23, 2024

Related U.S. Application Data
(60) Provisional application No. 63/356,173, filed on Jun. 28, 2022.

(51) Int. Cl. G16B 30/10; G16B 35/20; G16B 40/30

(52) U.S. Cl. CPC G16B 30/10; G16B 35/20; G16B 40/30

(57) ABSTRACT

The disclosure provides methods for predicting protospacer adjacent motif (PAM) sequences recognized by CRISPR-Cas proteins, libraries of CRISPR-Cas proteins having predicted PAM sequences, and uses thereof.

### 1. CROSS-REFERENCE TO RELATED APPLICATIONS

This application claims the priority benefit of U.S. provisional application No. 63/356,173, filed Jun. 28, 2022, the contents of which are incorporated herein in their entireties by reference thereto.

### 2. SEQUENCE LISTING

The instant application contains a Sequence Listing XML which has been submitted electronically and is hereby incorporated by reference in its entirety. Said Sequence Listing XML, created on Nov. 14, 2022, is named ALA-008WO_SL.xml and is 18,894 bytes in size.

### 3. BACKGROUND

Genome editing of mammalian cells using clustered regularly interspaced short palindromic repeats (CRISPR)-Cas (CRISPR-associated proteins) systems is a promising approach for treating genetic diseases (Doudna, 2020, Nature 578:229-236 (2020). Yet this technology is still limited by major constraints related to the limited number of known CRISPR-Cas systems active in mammalian cells. Protospacer adjacent motif (PAM) sequences, necessary for nuclease recognition and activity, are key in this context as they dictate the compatibility of each Cas protein towards specific genomic target sites. Molecular engineering of Cas9 proteins to relax PAM requirements has been explored (Collias & Beisel, 2021, Nat Commun 12 (1): 555), but this approach can impact the activity of the nucleases and still does not respond to all sequence requirements to treat genetic diseases. A substantial number of yet unexplored CRISPR-Cas systems exist in microbial genomes (Makarova et al., 2020, Nat. Rev. Microbiol. 18:67-83), and identification of PAM sequences for newly discovered CRISPR-Cas proteins is critical before such CRISPR-Cas proteins can be exploited as genome editing tools. However, experimentally determining PAM sequences for a large number of CRISPR-Cas proteins using existing methods is difficult, time consuming and costly.

Thus, new methods and systems for predicting PAM sequences of CRISPR-Cas proteins are needed.

### 4. SUMMARY

The disclosure provides methods for predicting CRISPR-Cas protein PAM sequences. The methods typically comprise performing PAM prediction for groups of CRISPR-Cas proteins clustered by percent amino acid sequence identity. Predicting a PAM sequence for the CRISPR-Cas proteins in a cluster typically includes a step of aligning spacer sequences from CRISPR arrays corresponding to the CRISPR-Cas proteins in the cluster to a database of viral genomes to identify putative protospacer sequences in the viral genomes that have matching or near matching sequences to the spacer sequences. As protospacer sequences in viral genomes are adjacent to PAM sequences, the sequences flanking the identified putative protospacer sequences should contain the PAM sequences for the CRISPR-Cas proteins in the cluster. The flanking sequences can be aligned to identify one or more conserved nucleotides in the upstream or downstream flanking sequences, where conserved nucleotides are indicative of the PAM sequence.

In one aspect, the disclosure provides a method for predicting a protospacer adjacent motif (PAM) sequence recognized by one or more CRISPR-Cas proteins in a CRISPR-Cas protein cluster, comprising:
a) mapping spacer sequences of one or more CRISPR arrays corresponding to one or more CRISPR-Cas proteins of the CRISPR-Cas protein cluster to a set of viral genome sequences to identify putative protospacer sequences in the viral genomes and their flanking upstream and downstream sequences in the viral genomes, wherein:
   i) mapping the spacer sequences to the viral genomes comprises aligning the spacer sequences to the viral genome sequences; and
   ii) the CRISPR-Cas protein cluster comprises a set of CRISPR-Cas protein sequences clustered by sequence identity;
b) for each spacer sequence mapped to one or more viral genome sequences, aligning the putative protospacer sequences and their flanking upstream and downstream viral genome sequences to generate a set of aligned putative protospacer and flanking sequences for each mapped spacer sequence; and
c) predicting a PAM sequence recognized by one or more CRISPR-Cas proteins in the cluster (e.g., all CRISPR-Cas proteins in the cluster) from the sets of aligned putative protospacer and flanking sequences.

In some embodiments, predicting a PAM sequence recognized by one or more CRISPR-Cas proteins in the cluster from the sets of aligned putative protospacer and flanking sequences comprises:
a) generating a consensus sequence from each set of aligned putative protospacer and flanking sequences to generate a set of consensus sequences, each consensus sequence comprising an upstream region and a downstream region;
b) calculating nucleotide frequencies at each nucleotide position in the set of consensus sequences for the upstream and downstream regions; and
c) identifying one or more conserved nucleotides in the upstream or downstream regions in the set of consensus sequences, wherein the presence of one or more conserved nucleotides indicates that the PAM sequence is positioned in the upstream or downstream consensus sequences.

An exemplary PAM prediction method of the disclosure is illustrated in FIG. 1. In FIG. 1, the CRISPR-Cas protein cluster is shown as having sequences for three CRISPR-Cas proteins, “Cas protein 1”, “Cas protein 2”, and “Cas protein 3”. It should be understood that the features illustrated in FIG. 1 are only exemplary. For example, a CRISPR-Cas cluster can have a few to many CRISPR-Cas protein sequences, for example at least one, at least 10, or at least 100 sequences, and/or up to 100, 1000, 2000, 4000, 5000, 10,000 or more than 10,000 sequences. FIG. 1 shows three spacers of CRISPR arrays corresponding to the CRISPR-Cas protein cluster (specifically, “Spacer 1”, “Spacer 2”, and “Spacer 3” from CRISPR arrays corresponding to Cas protein 1, Cas protein 2, and Cas protein 3, respectively). The spacer sequences can be mapped to a set of viral genome sequences. In this example, FIG. 1 shows that the spacer sequences map to viral genomes 1-9 (specifically, Spacer 1 maps to viral genomes 1-3; Spacer 2 maps to viral genomes 4-6; and Spacer 3 maps to viral genomes 7-9). By aligning the spacer sequences to the viral genomes, putative protospacer sequences in the viral genomes (illustrated in FIG. 1 as PP1 to PP9) and their flanking upstream and downstream sequences (illustrated in FIG. 1 as black bars flanking PP1 to PP9) can be identified. Then, the putative protospacer sequences and their flanking sequences can be aligned and consensus sequences generated for each set of alignments. In FIG. 1, PP1, PP2, and PP3 and their flanking sequences are aligned and consensus sequence C1 is generated from the alignment because Spacer 1 maps to the viral genome sequences corresponding to PP1, PP2, and PP3. Similarly, PP4, PP5, and PP6 and their flanking sequences are aligned and consensus sequence C2 is generated from the alignment because Spacer 2 maps to the viral genome sequences corresponding to PP4, PP5, and PP6; and PP7, PP8, and PP9 and their flanking sequences are aligned and a consensus sequence C3 is generated because Spacer 3 maps to the viral genome sequences corresponding to PP7, PP8, and PP9. Consensus sequences can be generated, for example, by taking the most frequent base at each position in an alignment. Columns in an alignment that contain many gaps (e.g., >30% gaps, >40% gaps, >50% gaps) can, in some embodiments, be discarded. Then, the PAM sequence for the CRISPR-Cas protein cluster can be predicted by identifying conserved nucleotides in the consensus sequences, as one or more conserved nucleotides in either the upstream or downstream flanking sequences are indicative of a PAM sequence. In FIG. 1, nucleotides conserved among consensus sequences C1, C2, and C3 are identified by asterisks. Conserved nucleotides can be identified, for example, by calculating nucleotide frequencies at each position for the consensus sequences and identifying positions where a particular nucleotide is present at a relatively high frequency. For example, a nucleotide can be considered conserved at a position if its frequency at the position is larger (e.g., significantly larger) than the frequencies of nucleotides at other positions in the upstream and downstream flanking sequences. In some embodiments, a sequence logo can be generated (e.g., for example by Logomaker (Tareen & Kinney, 2020, Bioinformatics 36:2272-2274)) to represent the nucleotide frequencies at each position.

The methods for predicting CRISPR-Cas protein PAM sequences are typically computer implemented. For example, a computer implemented method for predicting a PAM sequence can comprise executing, in a computer system having one or more processors coupled to a memory storing one or more computer readable instructions for execution by the one or more processors, the one or more computer readable instructions comprising instructions for:
a) mapping spacer sequences of one or more CRISPR arrays corresponding to one or more CRISPR-Cas proteins of the CRISPR-Cas protein cluster to a set of viral genome sequences to identify putative protospacer sequences in the viral genomes and their flanking upstream and downstream sequences in the viral genomes, wherein:
   i) mapping the spacer sequences to the viral genomes comprises aligning the spacer sequences to the viral genome sequences; and
   ii) the CRISPR-Cas protein cluster comprises a set of CRISPR-Cas protein sequences clustered by sequence identity;
b) for each spacer sequence mapped to putative protospacer sequences, aligning the protospacer sequences and their flanking upstream and downstream viral genome sequences to generate a set of aligned putative protospacer and flanking sequences for each mapped spacer sequence; and
c) predicting a PAM sequence recognized by one or more CRISPR-Cas proteins in the cluster from the sets of aligned putative protospacer and flanking sequences.

Implementing the PAM prediction methods of the disclosure by computer allows for the generation of libraries of CRISPR-Cas proteins with predicted PAM sequences (referred to herein as “CRISPR-Cas protein libraries of the disclosure” or “libraries of the disclosure” for convenience), e.g., libraries of 1,000 or more, 10,000 or more, 50,000 or more, or even 100,000 or more CRISPR-Cas proteins, e.g., Cas9 proteins, with predicted PAM sequences. For example, an initial set of 1,000 or more CRISPR-Cas protein sequences (e.g., previously uncharacterized CRISPR-Cas protein sequences, for example Cas9 protein sequences) can be clustered by percent sequence identity to generate a set of clusters. PAMs can be predicted for the clusters and, by extension, the individual CRISPR-Cas proteins whose amino acid sequences make up the clusters.

Further exemplary features of the methods of predicting PAM sequences are described in Section 6.2 and specific embodiments 1 to 100, infra.

The disclosure further provides systems configured to predict PAM sequences according to the PAM prediction methods of the disclosure. The systems typically comprise one or more processors coupled to a memory storing one or more computer readable instructions for performing steps of the method for execution by the one or more processors.

The disclosure further provides tangible, non-transitory computer-readable media comprising instructions executable by a processor, where the instructions include instructions for executing a PAM prediction method of the disclosure.

The disclosure further provides tangible, non-transitory computer-readable media comprising a library of CRISPR-Cas protein sequences with predicted PAM sequences obtained or obtainable by the PAM prediction methods of the disclosure.

Further exemplary features of systems and tangible, non-transitory computer-readable media of the disclosure are described in Section 6.3 and specific embodiments 101 to 104, infra.

The disclosure further provides uses of CRISPR-Cas protein libraries of the disclosure.

In one aspect, the disclosure provides methods of selecting a CRISPR-Cas protein for editing a genomic sequence of interest (e.g., a gene variant associated with a disease). Such methods can comprise (a) identifying, in a library of the disclosure, a predicted PAM whose sequence is present in the genomic sequence of interest, and (b) selecting a CRISPR-Cas protein from the library whose predicted PAM is present in the genomic sequence of interest.

In another aspect, the disclosure provides methods of selecting a CRISPR-Cas protein for editing a genomic sequence comprising a PAM of interest, e.g., for editing in the vicinity of the PAM of interest (e.g., for cutting genomic DNA within 30 nucleotides, within 20 nucleotides, within 10 nucleotides, or within 5 nucleotides of the PAM sequence), the methods comprising selecting a CRISPR-Cas protein from a CRISPR-Cas library of the disclosure whose predicted PAM sequence corresponds to the PAM of interest. A predicted PAM sequence “corresponds to” a PAM of interest when the predicted PAM sequence and the PAM of interest are the same (e.g., both are “AGG”) or, when the predicted PAM sequence allows for variability at one or more positions (e.g., “NGG,” where N can be any nucleobase) one of the nucleotide sequences defined by predicted PAM sequence is the same as the sequence of the PAM of interest. For example, a predicted PAM sequence “NGG,” where N can be any nucleobase, corresponds to a PAM of interest having the sequence AGG, TGG, CGG, or GGG. A CRISPR-Cas protein can be selected for editing of a genomic sequence having a PAM created by a mutation, e.g., a disease-causing mutation, for example a mutation that is associated with an autosomal dominant disease. By targeting a PAM that is present in a mutant allele but not a wild-type allele, editing of genomic DNA can be performed in an allele-specific manner. One or more CRISPR-Cas proteins selected from the library can be evaluated for editing ability in vitro (e.g., by evaluating indel formation), and candidates having promising in vitro editing activity can be selected for further evaluation, for example further in vitro and/or in vivo studies.

In another aspect, the disclosure provides methods of predicting a PAM sequence recognized by a CRISPR-Cas protein of interest, for example a previously uncharacterized CRISPR-Cas protein. Such methods can comprise identifying a CRISPR-Cas protein in a library of the disclosure having relatively high (or having the highest) amino acid sequence identity with the CRISPR-Cas protein of interest (e.g., greater than 95%, greater than 96%, greater than 97%, greater than 98%, greater than 99%, or 100% identity), and predicting that the PAM sequence of the CRISPR-Cas protein of interest is the same as the PAM sequence of the CRISPR-Cas protein in the library.

In another aspect, the disclosure provides methods for selecting a CRISPR-Cas protein for editing a genomic sequence of interest, comprising (a) identifying, in a CRISPR-Cas library of the disclosure, PAM sequences in the library whose sequences are present in the genomic sequence of interest; (b) identifying one or more CRISPR-Cas proteins from the library whose predicted PAM sequence(s) are present in the genomic sequence of interest; (c) evaluating the ability of the one or more CRISPR-Cas proteins to edit the genomic sequence; and (d) selecting a CRISPR-Cas protein from the one or more CRISPR-Cas proteins following the evaluation.

In another aspect, the disclosure provides methods for selecting a CRISPR-Cas protein for editing a genomic sequence comprising a PAM of interest (e.g., a PAM sequence created by a mutation, for example a disease-causing mutation), comprising (a) identifying, in a CRISPR-Cas library of the disclosure, one or more CRISPR-Cas proteins whose predicted PAM sequences correspond to the PAM sequence of interest; (b) evaluating the ability of the one or more CRISPR-Cas proteins to edit the genomic sequence; and (c) selecting a CRISPR-Cas protein from the one or more CRISPR-Cas proteins following the evaluation.

In another aspect, the disclosure provides methods for selecting a genomic sequence having a pathogenic mutation for editing with a CRISPR-Cas protein, comprising (a) identifying, in a CRISPR-Cas library of the disclosure, a PAM sequence associated with a pathogenic mutation; and (b) selecting a genomic sequence that includes that PAM sequence for editing with a CRISPR-Cas protein, for example a CRISPR-Cas protein in the library whose PAM corresponds to the PAM sequence associated with the pathogenic mutation.

In another aspect, the disclosure provides methods of designing a guide RNA (gRNA) molecule for a CRISPR-Cas protein in a CRISPR-Cas protein library of the disclosure. The gRNAs can be designed, for example, for a CRISPR-Cas protein selected by a method of the disclosure to edit a genomic sequence of interest, for example, a genomic sequence having a PAM of interest, as described herein.

In another aspect, the disclosure provides methods for editing a genomic sequence. Such methods can comprise contacting a cell (e.g., in vivo or ex vivo) with a system comprising a CRISPR-Cas protein selected according to a method described herein and a gRNA for editing the genomic sequence. For example, the method can comprise contacting a cell with one or more nucleic acids encoding the CRISPR-Cas protein and a gRNA targeting a genomic sequence adjacent to the PAM sequence of the CRISPR-Cas protein.

Further exemplary features of uses of CRISPR-Cas protein libraries of the disclosure are described in Section 6.4 and specific embodiments 105 to 142, infra.

### 5. BRIEF DESCRIPTION OF THE FIGURES

FIG. 1 illustrates an exemplary PAM prediction method of the disclosure.

FIG. 2 shows a schematic of the pipeline used to generate PAM predictions in Example 1.

FIG. 3 shows predicted PAM sequences for Cas9 proteins with in vitro verified PAM sequences. Sequence identity between the Cas9 proteins with known PAMs and the Cas9 with predicted PAMs in the dataset of Example 1 is also shown.

FIGS. 4A-4D both show predicted and in vitro determined PAMs for selected Cas9 variants described in Gasiunas et al., 2020, Nat. Commun. 11:5512. For each indicated Cas9 the in vitro (IVT) derived (left) and in silico predicted (right) logos are shown. Percentages of sequence identity and measure of the distance between predicted and true PAMs are reported for each Cas9.

FIG. 5 shows a phylogenetic tree of selected Cas9 proteins commonly used for genome editing applications (CjCas9, Nm1Cas9, SaCas9, SpCas9) and newly characterized Cas9 proteins (SuCas9, BsCas9, Al2Cas9, Al1Cas9). Protein alignments: grey aligned protein sequences, black conserved sequences, colored conserved domains. Length: number of amino acids.

FIG. 6 shows predicted and in vitro determined PAMs for selected Cas9 variants from the dataset of Example 1.

FIG. 7 shows a boxplot of distance between in vitro and predicted PAMs for evaluated Cas9 orthologs. Most orthologs have a low (<2 bits) PAM distance.

FIG. 8 shows the fraction of Cas9 clusters of Example 1 with more than 10 mapped spacers and a predicted PAM, for each Cas9 subtype.

FIG. 9 shows a hierarchical clustering tree generated from pairwise PAM distances, with annotated PAM clusters and consensus PAM for each cluster (Example 1). Different groups of PAMs with conserved bases in specific positions were identified.

FIG. 10 shows associations between the 10 most abundant PAM clusters and the Cas9 phylogenetic tree (Example 1).

FIG. 11 illustrates the mode of inheritance of mutations in ClinVar that can be potentially targeted by Cas9 proteins in the dataset of Example 1.

FIG. 12 shows the predicted and in vitro determined PAMs for PrCas9.

FIG. 13 shows the organization of the PrCas9 CRISPR-Cas locus.

FIG. 14 shows the predicted protein domain organization of PrCas9.

FIG. 15 shows the structure of a single guide RNA (sgRNA) of PrCas9 (spacer: SEQ ID NO: 18; sgRNA scaffold: SEQ ID NO: 19).

FIG. 16 shows a PAM heatmap for PrCas9, showing the nucleotide preference for positions 2, 3, 5 and 6 (no nucleotide preference is present at other positions). The preferred PAM is NRVNRT, V=A, C or G; R=G or A.

FIG. 17 shows editing activity of PrCas9 in mammalian cells using an EGFP disruption assay by measuring the fluorescence of U2OS cells stably expressing EGFP and transfected with SpCas9, PrCas9 or with control plasmids (Ctr).

FIG. 18 shows editing activity (% indels) in RHO wild-type or carrying the P23H mutation.

### 6. DETAILED DESCRIPTION

The present disclosure addresses the need in the art for systems and methods for predicting PAM sequences of CRISPR-Cas proteins, e.g., for predicting PAM sequences for previously uncharacterized CRISPR-Cas proteins.

### 6.1. Definitions

Unless defined otherwise herein, all technical and scientific terms used herein have the same meaning as commonly understood by one of ordinary skill in the art. Various scientific dictionaries that include the terms included herein are well known and available to those in the art.

As used herein, the singular forms “a”, “an” and “the” include plural referents unless the content and context clearly dictates otherwise. Thus, for example, reference to “a CRISPR-Cas protein cluster” includes a combination of two CRISPR-Cas protein clusters, a combination of three CRISPR-Cas protein clusters, and the like.

Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

CRISPR-Cas protein cluster refers to a set of CRISPR-Cas protein sequences grouped (or clustered) according to sequence identity level (e.g., a user-defined sequence identity level). Algorithms for clustering protein sequences are known in the art and include, for example, UCLUST, e.g., version 11.0.667, (Edgar, 2010, Bioinformatics 26:2460-2461) and CD-HIT (Weizhong & Godzik, 2006, Bioinformatics 22 (13): 1658-1659). Some clustering algorithms, such as UCLUST, define a cluster by a sequence known as the centroid or representative sequence, and each sequence in the cluster has a percent identity to the centroid sequence above a user-defined percent identity threshold. For example, in some embodiments the user-defined percent identity threshold can be set at 100%, 99%, 98%, 97%, 96% or 95%.

A CRISPR-Cas locus refers to a prokaryotic genomic region having (i) a sequence encoding a CRISPR-Cas protein and (ii) a CRISPR array having spacer sequences, which are short sequences that originate from and correspond to viral DNA sequences called protospacers. An exemplary CRISPR-Cas locus is illustrated in FIG. 13. CRISPR arrays are composed of repeat sequences flanking spacer sequences. A CRISPR array can have at least one spacer, each flanked by two repeats. CRISPR arrays are typically located in the vicinity of cas genes but are sometimes located distant to cas genes (Butiuc-Keul et al., 2022, Microb Physiol 32:2-17; Shmakov et al., 2020, CRISPR J. 3 (6): 535-549. Exemplary tools for identifying and annotating CRISPR-Cas loci and CRISPR arrays include CRISPRCasTyper (Russel et al., 2020, CRISPR J. 3:462-469) and CRISPRDetect (Biswas et al., 2016, BMC Genomics 17:356). A CRISPR array “corresponds to” a CRISPR-Cas protein when the sequence encoding the CRISPR-Cas protein and the CRISPR array are from the same genome.

Guide RNA molecule (gRNA) refers to an RNA capable of forming a complex with a CRISPR-Cas protein and which can direct the CRISPR-Cas protein to a target DNA. gRNAs typically comprise a targeting sequence of 15 to 30 nucleotides in length in length that is complementary to a target genomic nucleotide sequence. The targeting sequence for a Type II Cas protein (e.g., a Cas9 protein) can be positioned 5′ of a crRNA scaffold to form a full crRNA. The crRNA can be used with a tracrRNA to effect cleavage of a target genomic sequence. gRNAs of the disclosure are in some embodiments single guide RNAs (sgRNAs), which typically comprise a crRNA sequence fused to a tracrRNA sequence.

Identical or percent identity, in the context of two or more nucleic acids or polypeptide sequences, refer to two or more sequences or subsequences that are the same or have a specified percentage of amino acid residues or nucleotides that are the same as measured using a sequence comparison algorithm. Alignment for purposes of determining percent sequence identity can be achieved in various ways that are within the skill in the art, for instance, using publicly available computer software such as BLAST, ALIGN, ALIGN-2 or Megalign (DNASTAR) software. Appropriate parameters for measuring alignment, including any algorithms needed to achieve maximal alignment over the full-length of the sequences being compared can be determined by known methods.

One example of an algorithm that is suitable for determining percent sequence identity and sequence similarity is the BLAST algorithm, which is described in Altschul et al., 1990, J. Mol. Biol. 215:403-410. Software for performing BLAST analyses is publicly available, for example, through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

Protospacer adjacent motif (PAM) refers to a DNA sequence downstream (e.g., immediately downstream) (e.g., in the case of Type II Cas proteins such as Cas9) or upstream (e.g., immediately upstream) (e.g., in the case of Type V Cas proteins such as Cas12a) of a target sequence recognized by a CRISPR-Cas protein. A PAM sequence is necessary for a CRISPR-Cas protein to bind and cut target genomic DNA.

Putative protospacer refers to a nucleotide sequence in a viral genome that aligns with a spacer sequence with no or only a small number of mismatches or gaps. In some embodiments, a nucleotide sequence in a viral genome is identified as a putative protospacer if the spacer aligns with the nucleotide sequence in the viral genome with no more than four nucleotide mismatches or gaps. In some embodiments, a nucleotide sequence in a viral genome is identified as a putative protospacer if the spacer aligns with the nucleotide sequence in the viral genome with no more than three nucleotide mismatches or gaps. In some embodiments, a nucleotide sequence in a viral genome is identified as a putative protospacer if the spacer aligns with the nucleotide sequence in the viral genome with no more than two nucleotide mismatches or gaps. In some embodiments, a nucleotide sequence in a viral genome is identified as a putative protospacer if the spacer aligns with the nucleotide sequence in the viral genome with no more than one nucleotide mismatch or gap. In some embodiments, a nucleotide sequence in a viral genome is identified as a putative protospacer if the spacer aligns with the nucleotide sequence in the viral genome with no nucleotide mismatches or gaps.

Algorithms for aligning sequences are known in the art, for example BLASTN (Altschul et al., 1990, J. Mol. Biol. 215:403-410).

### 6.2. Methods for CRISPR-Cas Protein PAM Identification

In certain aspects, the disclosure provides methods for predicting a PAM sequence recognized by one or more (e.g., some or all) CRISPR-Cas proteins in a CRISPR-Cas protein cluster. Predicting a PAM sequence can comprise (a) mapping spacer sequences of CRISPR arrays corresponding to a CRISPR-Cas protein cluster to a set of viral genome sequences to identify putative protospacer sequences in the viral genomes and their flanking upstream and downstream sequences in the viral genomes; (b) for each spacer sequence mapped to one or more viral genome sequences, aligning the putative protospacer sequences and their flanking upstream and downstream viral genome sequences to generate a set of aligned putative protospacer and flanking sequences for each mapped spacer sequence; and (c) predicting a PAM sequence recognized by one or more CRISPR-Cas proteins in the cluster from the sets of aligned putative protospacer and flanking sequences.

The method can be computer implemented. Computer implemented methods can comprise executing, in a computer system having one or more processors coupled to a memory storing one or more computer readable instructions for execution by the one or more processors, the one or more computer readable instructions comprising instructions for steps (a)-(c) as described in the preceding paragraph.

### 6.2.1. CRISPR-Cas Protein Clusters

CRISPR-Cas protein clusters can be generated from an initial set of CRISPR-Cas proteins (e.g., an initial set of CRISPR-Cas proteins having at least 10,000, at least 50,000, at least 75,000, at least 90,000 and/or up to 1 million, up to 750,000, up to 500,000, up to 200,000, or up to 100,000 CRISPR-Cas protein sequences) by use of a clustering algorithm that clusters amino acid sequences by percent identity. For example, in some embodiments UCLUST (Edgar, 2010, Bioinformatics 26:2460-2461) or CD-HIT (Weizhong & Godzik, 2006, Bioinformatics 22 (13): 1658-1659) are used. In some embodiments, UCLUST is used to cluster CRISPR-Cas proteins at the 95%, 96%, 97%, 98%, 99%, or 100% identity level. In some embodiments, UCLUST is used to cluster CRISPR-Cas proteins at the 98% identity level. PAM prediction methods of the disclosure comprise a step of generating one or more CRISPR-Cas protein clusters. Alternatively, PAM prediction can be performed using one or more previously generated CRISPR-Cas protein clusters.

An initial set of CRISPR-Cas proteins used to make a CRISPR-Cas protein cluster can be obtained from bacterial and/or archael genomes. For example, genomes (e.g., at least 100,000, 200,000, 500,000, 800,000, 1 million, or more, and/or up to 2 million genomes) can be retrieved from a database, for example NCBI, and an algorithm can be used to identity CRISPR-Cas loci, for example CRISPR-Cas9 loci. Algorithms for identifying CRISPR-Cas loci are known in the art and include, for example, CRISPRCasTyper (Russel et al., 2020, CRISPR J. 3:462-469). PAM prediction methods of the disclosure comprise a step of generating an initial set of CRISPR-Cas protein sequences, which is then used to generate one or more CRISPR-Cas protein clusters. Alternatively, a pre-existing set of CRISPR-Cas protein sequences can be used to generate one or more CRISPR-Cas protein clusters.

The methods of the disclosure can be performed using various types of CRISPR-Cas protein sequences. For example, the CRISPR-Cas proteins in a CRISPR-Cas protein cluster can comprise or consist of Class II Cas proteins, e.g., Type II Cas proteins (e.g., Cas9), Type V Cas proteins (e.g., Cas12a), or Type VI Cas proteins (e.g., Cas13) (for reviews of Class II Cas proteins, see Makarova et al., 2020, Nat Rev Microbiol 18 (2): 67-83; Tong et al., 2020, Front Cell Dev Biol. 8:622103; and Chylinksi et al., 2014 Nucleic Acids Res. 42 (10): 6091-6105, the contents of which are incorporated herein by reference in their entireties). Type II Cas proteins include, for example, Type II-A, Type II-B, and Type II-C. Type V Cas proteins include, for example, Type V-A, Type V-B, Type V-C, Type V-D, Type V-E, Type V-F, Type V-G, Type V-H, Type V-I, Type V-J, and Type V-K. In some embodiments, the CRISPR-Cas proteins are Type II Cas proteins, for example Cas9 proteins. In other embodiments, the CRISPR-Cas proteins are Type V Cas proteins (e.g., Cas12a).

An initial set of CRISPR-Cas proteins (e.g., Type II Cas proteins such as Cas9 proteins) contains protein sequences having a minimum and/or maximum sequence length. For example, a minimum length can be 150, 200, 400, 500, 600, 700, 800, 900, 950, 1000, 1100, 1200 or 1300 amino acids. A maximum length can be, for example, 2200, 2100, 2000, 1900, 1800, 1700, 1600, 1500, 1400, 1300, 1200 or 1100 amino acids. Selecting a maximum length, for example of 1100 amino acids, can be used, for example, to limit a set of CRISPR-Cas proteins to those which can be packaged together with a gRNA in a single AAV vector genome. In other embodiments, an initial set of CRISPR-Cas proteins is not limited to those having a particular amino acid sequence length.

The number of CRISPR-Cas protein sequences in a CRISPR-Cas protein cluster can vary, for example depending on the number of CRISPR-Cas protein sequences in an initial set of CRISPR-Cas proteins subjected to clustering and/or depending on how divergent the sequences in the initial set of CRISPR-Cas protein are from each other. A CRISPR-Cas protein cluster can contain, for example, at least 1, at least 10, or at least 100 CRISPR-Cas protein sequences.

While the PAM prediction methods can be performed with a single CRISPR-Cas protein cluster, the method can be performed using more than one cluster, for example 100 clusters or more, 1000 clusters or more, 5000 cluster or more 10,000 clusters or more. In some embodiments, the methods are performed on up to 100,000 clusters.

To increase reliability of PAM predictions, clusters having a low number of mapped spacers can in some embodiments be discarded. For example, in some embodiments, cluster having fewer than 50, fewer than 25, or fewer than 10 mapped spacers are discarded. In some embodiments, clusters having fewer than 10 mapped spacers are discarded. In some embodiments, clusters having fewer than 5 mapped spacers are discarded.

In some embodiments, a PAM prediction method of the disclosure is repeated using a different CRISPR-Cas protein cluster(s) and/or a different set of viral genomes from those used initially. Repeating the method using a different CRISPR-Cas protein cluster(s) and/or a different set of viral genomes can be useful, for example, to update a CRISPR-Cas protein library with additional CRISPR-Cas protein sequences and/or to update a CRISPR-Cas protein library after a new set of viral genomes becomes available.

### 6.2.2. Mapping Spacer Sequences to Viral Genomes

The set of viral genome sequences can comprise, for example, a set of phage genomes, for example phage genomes of the human microbiome. Various sources of viral genomes can be used, for example the Gut Phage Database and the Metagenomic Gut Virus catalog (Nayfach et al., 2021 Nat. Microbiol. 6:960-970). Viral genomes can also be assembled de novo, for example using BLASTN.

In some embodiments, the set of viral genomes contains at least 100,000 viral genomes, at least 200,000 viral genomes at least 300,000 viral genomes and/or up to 1 million viral genomes, up to 750,000 viral genomes, up to 500,000 viral genomes, or up to 400,000 viral genomes.

Spacer sequences of CRISPR arrays corresponding to one or more CRISPR-Cas proteins of a CRISPR-Cas protein cluster can be mapped to a set of viral genome sequences by performing sequence alignments of a given spacer sequence to the viral genome sequences, for example by using a sequence alignment algorithm such as BLASTN. A given spacer sequence can be considered mapped to a viral genome sequence when, for example, the spacer sequence aligns over its full length to a viral genome sequence with a near-perfect or perfect nucleotide match. In some embodiments, a spacer is considered mapped to a viral genome sequence when there are no more than four nucleotide mismatches or gaps (e.g., 4, 3, 2, 1, or 0 nucleotide mismatches or gaps) in an alignment of the full-length spacer sequence and a viral genome sequence.

The viral genome sequences to which a given spacer sequence is mapped can be identified as putative protospacer sequences. Following identification of putative protospacer sequences corresponding to a given spacer sequence, the viral genomic sequences flanking the putative protospacer sequences both upstream and downstream can be retrieved. The upstream and downstream sequences are retrieved because true CRISPR-Cas protospacer sequences are adjacent to a PAM sequence. Because the PAM is adjacent to the protospacer, it is only necessary to retrieve a relatively small number of flanking nucleotides. In some embodiments, up to 40 nucleotides upstream and up to 40 nucleotides downstream of the putative protospacer sequences are retrieved. In some embodiments, up to 30 nucleotides upstream and up to 30 nucleotides downstream of the putative protospacer sequences are retrieved. In some embodiments, up to 20 nucleotides upstream and up to 20 nucleotides downstream of the putative protospacer sequences are retrieved. In some embodiments, up to 10 nucleotides upstream and up to 10 nucleotides downstream of the putative protospacer sequences are retrieved.

6.2.3. Predicting PAM Sequences from Putative Protospacer Sequences and Flanking Sequences

Putative protospacer sequences and their flanking upstream and downstream sequences can be aligned to generate a set of aligned putative protospacer and flanking sequences. For example, a multiple sequence alignment tool such as MUSCLE (Edgar, 2004, Nucleic Acids Research 32 (5): 1792-97; Edgar, 2004, BMC Bioinformatics 5:113) or MAFFT (Katoh et al., 2002, Nucleic Acids Research 30 (14): 3059-3066) can be used. In some embodiments, MUSCLE is used. In other embodiments, MAFFT is used.

As protospacer sequences are adjacent to PAM sequences in viral genomes, the sets of aligned putative protospacer and flanking sequences generated for the different spacer sequences can be used to predict the PAM sequence for the CRISPR-Cas proteins of a CRISPR-Cas protein cluster. For example, a consensus sequence can be generated from each set of aligned putative protospacer and flanking sequences (each set corresponding to a single spacer), for example by taking the most frequent base at each position in the alignment, discarding columns composed of many or composed mostly of (e.g., >50%) gaps. The spacer sequence can be aligned to the consensus sequence to define the upstream and downstream regions of the consensus sequence.

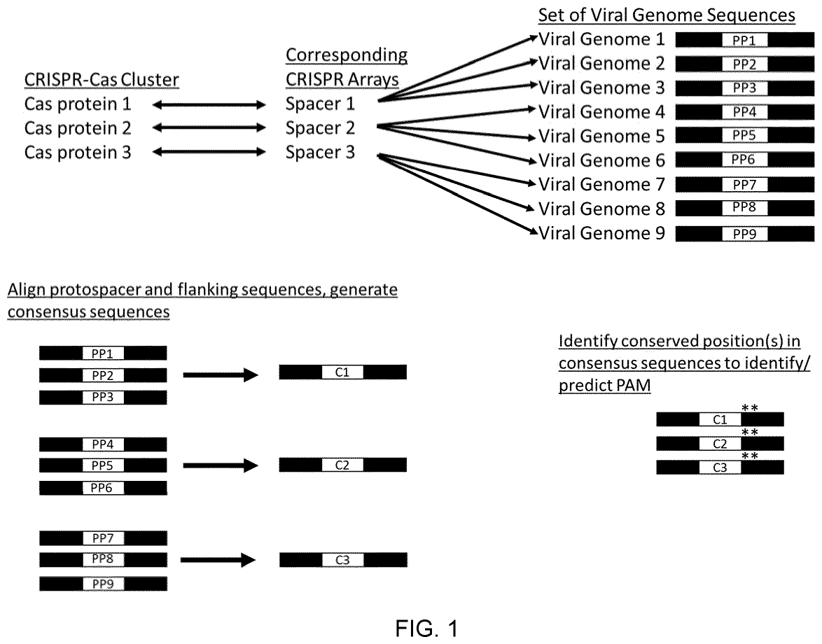

A consensus sequence can be generated from each set of aligned putative protospacer and flanking sequences, for example as illustrated in FIG. 1. The nucleotide frequencies at each position in the upstream and downstream flanking sequences in the consensus sequences can be computed to identity one or more conserved nucleotides, which are indicative of a PAM sequence for a CRISPR-Cas protein cluster. In other words, a PAM sequence for a cluster (and, by extension, each CRISPR-Cas protein whose sequence is in the cluster) can be predicted by analyzing the nucleotide frequencies at nucleotide positions in the upstream and downstream sequences to identify nucleotides present at a relatively high frequency (e.g., outliers), because nucleotides present at a relatively high frequency (e.g., outliers) are likely to correspond to nucleotides of the PAM. In some embodiments, a sequence logo can be generated from the nucleotide frequencies at positions in the upstream and downstream flanking sequences, which can provide a graphical representation of the sequence conservation. In some embodiments, a nucleotide in a sequence logo can be considered a conserved nucleotide when it has more than one bit of information. In some embodiments, a nucleotide is considered conserved when it has more than one bit of information and its bit level is larger than the median bit level in both flanking regions plus 1.5 times the interquartile range of bit levels in both flanking regions. Tools for generating sequence logos include Logomaker (Tareen & Kinney, 2020, Bioinformatics 36:2272-2274). In some embodiments, the methods comprise generating a report with the sequence logo in a computerized system.

The methods can further comprise a step of generating a report with the predicted PAM for a CRISPR-Cas protein cluster. The report can include, for example, identifying information for one or more CRISPR-Cas proteins (e.g., CRISPR-Cas protein name and/or ID number and/or CRISPR-Cas protein amino acid sequences and/or species information) and their corresponding predicted PAM sequences. The information in a report can be included in a CRISPR-Cas protein library of the disclosure.

Following prediction of a PAM for a CRISPR-Cas protein cluster, the predicted PAM can optionally be validated, for example by an in vitro assay, for example as described in Example 1, using one or more CRISPR-Cas proteins of the cluster. To validate a PAM in vitro, tracrRNA sequences for the one or more CRISPR-Cas proteins to be used for PAM validation can be determined so that an appropriate gRNA for the in vitro assay can be designed. tracrRNA sequences can be identified computationally, for example, as described in Example 1. tracrRNA sequences, which contain an anti-repeat and a Rho-independent terminator (RIT), can be identified by aligning CRISPR repeats to sequences flanking a CRISPR-Cas locus (e.g., up to 1000 nucleotides) (for example, using BLASTN) to identify putative anti-repeats. RITs can be predicted using RNIE software (Gardner et al., 2011, Nucleic Acids Res. 39:5845-5852). Other methods for predicting tracrRNAs can also be used, for example as described in Chyour and Brown 2019, RNA Biol. 16 (4): 423-434 or Dooley et al., 2021 CRISPR J. 4 (3): 438-447, each of which is incorporated herein by reference in its entirety.

When PAMs are predicted for a plurality of CRISPR-Cas protein clusters, the CRISPR-Cas protein sequences of the CRISPR-Cas protein clusters can be clustered according to their predicted PAM sequences to generate PAM clusters. PAM clustering can be performed, for example, by computing an all-to-all PAM prediction distance matrix using a clustering tool such as usearch (Edgar, 2010, Bioinformatics 26:2460-2461). Hierarchical clustering can be performed on a PAM cluster, for example as described in Example 1. PAM clustering can be useful for measuring the diversity of a library, and for studying the hierarchical relationship between CRISPR-Cas proteins in a library.

### 6.3. Systems and Computer-Readable Media

The present disclosure further provides a system configured to predict PAM sequences for CRISPR-Cas proteins according to any one of the computer implemented methods disclosed herein. The system typically comprises one or more processors coupled to a memory storing one or more computer readable instructions for execution by the one or more processors.

In some embodiments, the one or more computer readable instructions can comprise instructions for performing one, more than one, or all steps of a PAM prediction method described herein and, optionally, one or more computer implemented steps of a method described in Section 6.4 performed subsequent to PAM prediction.

The systems and methods disclosed herein can be incorporated into a software tool accessible to researchers, e.g., researchers seeking to develop a treatment for genetic diseases. For example, the software tool may be incorporated at least partially into a computer system used by a researcher or other user. The computer system may receive a PAM sequence or other sequence information, for example sequence information relating to a disease-causing (pathogenic) mutation or a genomic sequence associated with a genetic disease. For example, the data may be input by the researcher and/or may be received over a network, such as the Internet, from another source capable of accessing and providing such data. The data may be transmitted via a network or other system for communicating the data, directly into the computer system, or a combination thereof. The software tool may use the data to identify one or more CRISPR-Cas proteins whose predicted PAM corresponds to a PAM sequence input or received, or whose predicted PAM is present in a genomic sequence input or received. If the data input by the researcher and/or received over a network relates to a disease-causing mutation, the software tool may use the data to determine whether the disease-causing mutation creates a genomic sequence matching a PAM sequence predicted for one or more CRISPR-Cas proteins in a library, and provide information to the user relating to the one or more CRISPR-Cas proteins. For example, the software tool may provide a recommendation of one or more CRISPR-Cas proteins for editing the genomic sequence in the vicinity of the PAM, and, optionally, provide a recommendation for one or more gRNA sequences.

Alternatively, the software tool may be provided as part of a web-based service or other service, e.g., a service provided by an entity that is separate from the researcher. The service provider may, for example, operate the web-based service and may provide a web portal or other web-based application (e.g., run on a server or other computer system operated by the service provider) that is accessible to researchers or other users via a network or other methods of communicating data between computer systems. For example, data relating to a disease-causing mutation or genomic sequence of interest may be provided to the service provider, and the service provider may identify one or more CRISPR-Cas proteins whose PAM corresponds to a genomic sequence created by the mutation or whose PAM corresponds to a sequence in the genomic sequence of interest. Then, the web-based service may transmit one or more CRISPR-Cas protein sequences and, optionally, one or more gRNA sequences to the researcher's computer system or display the CRISPR-Cas protein sequence(s) and, optionally gRNA sequence information to the researcher.

One or more of the steps described herein may be performed by one or more human operators (e.g., a researcher, an employee of the service provider providing the web-based service, other user, etc.), or one or more computer systems used by such human operator(s), such as a desktop or portable computer, a workstation, a server, a personal digital assistant, etc. The computer system(s) may be connected via a network or other method of communicating data.

Reports may also be generated using a combination of any of the features set forth herein. More broadly, any aspect set forth in any embodiment may be used with any other embodiment set forth herein.

In further aspects, the disclosure provides tangible, non-transitory computer-readable media that comprise instructions for one or more of the computer implemented methods described herein, and provides tangible, non-transitory computer-readable media that comprise CRISPR-Cas protein sequences with predicted PAM sequences obtained or obtainable by the methods described herein. In some embodiments, the disclosure provides tangible, non-transitory computer-readable media containing information relating to a CRISPR-Cas protein library of the disclosure, for example, CRISPR-Cas protein sequences and predicted and/or in vitro validated PAM sequences for the CRISPR-Cas protein sequences. Examples of non-transitory computer media include internal disks (e.g., hard drives) and removable disks (e.g., flash drives, DVDs, CDROMs, etc.).

### 6.4. Uses of Libraries of CRISPR-Cas Proteins with Predicted PAMs

In one aspect, the disclosure provides methods of identifying a PAM sequence recognized by a CRISPR-Cas protein comprising predicting a PAM sequence recognized by the CRISPR-Cas protein according to a PAM prediction method described herein (e.g., by a computer implemented method described herein) and, subsequently, validating the predicted PAM in vitro, thereby identifying the PAM sequence for the CRISPR-Cas protein. An exemplary method for validating a PAM in vitro is described in Example 1.

In another aspect, the disclosure provides methods of selecting a CRISPR-Cas protein for editing a genomic sequence of interest (e.g., a gene associated with a disease). Such methods can comprise (a) identifying, in a library of the disclosure, a predicted PAM whose sequence is present in the genomic sequence of interest, and (b) selecting a CRISPR-Cas protein from the library whose PAM is present in the genomic sequence of interest. In some embodiments, step (a) and/or step (b) are computer implemented.

In another aspect, the disclosure provides methods of selecting a CRISPR-Cas protein for editing a genomic sequence comprising a PAM of interest, e.g., for editing in the vicinity of the PAM of interest (e.g., for cutting genomic DNA within 30 nucleotides, within 20 nucleotides, within 10 nucleotides, or within 5 nucleotides of the PAM sequence). Such methods can comprise selecting a CRISPR-Cas protein from a CRISPR-Cas library of the disclosure whose predicted PAM sequence corresponds to the PAM of interest. For example, a CRISPR-Cas protein can be selected for editing of a genomic sequence having a PAM created by a mutation, e.g., a disease-causing mutation (also referred to as a pathogenic mutation) mutation. For example, the mutation can be a mutation associated with an autosomal dominant disease, such as a mutation in a RHO gene that causes retinitis pigmentosa (e.g., P23H). By targeting a PAM that is present in a mutant allele but not a wild-type allele, editing of genomic DNA can be performed in an allele-specific manner. The step of selecting the CRISPR-Cas protein from the library can be computer implemented. One or more CRISPR-Cas proteins selected from the library can be evaluated for editing ability in vitro (e.g., by evaluating indel formation), and candidates having promising in vitro editing activity can be selected for further evaluation.

In another aspect, the disclosure provides methods of predicting a PAM sequence recognized by a CRISPR-Cas protein of interest, for example a previously uncharacterized CRISPR-Cas protein. Such methods can comprise identifying a CRISPR-Cas protein in a library of the disclosure having relatively high (or highest) amino acid sequence identity with the CRISPR-Cas protein of interest (e.g., greater than 95%, greater than 96%, greater than 97%, greater than 98%, greater than 99%, or 100% identity), and predicting that the PAM sequence of the CRISPR-Cas protein of interest is the same as the PAM sequence of the CRISPR-Cas protein in the library. In some embodiments, such methods are computer implemented. After a PAM is predicted for the CRISPR-Cas protein of interest, the prediction can optionally be validated in vitro.

In another aspect, the disclosure provides methods for selecting a CRISPR-Cas protein for editing a genomic sequence of interest, comprising (a) identifying, in a CRISPR-Cas library of the disclosure, PAM sequences in the library whose sequences are present in the genomic sequence of interest; (b) identifying one or more CRISPR-Cas proteins from the library whose predicted PAM sequence(s) are present in the genomic sequence of interest; (c) evaluating the ability of the one or more CRISPR-Cas proteins to edit the genomic sequence (e.g., by an in vitro gene editing assay, for example as described in Example 1); and (d) selecting a CRISPR-Cas protein from the one or more CRISPR-Cas proteins following the evaluation. The selected CRISPR-Cas protein(s) can be, for example, the CRISPR-Cas protein(s) having the highest editing activity and/or highest fidelity in an in vitro gene editing assay. In some embodiments, steps (a) and/or (b) are computer implemented.

In another aspect, the disclosure provides methods for selecting a CRISPR-Cas protein for editing a genomic sequence comprising a PAM of interest, comprising (a) identifying, in a CRISPR-Cas library of the disclosure, one or more CRISPR-Cas proteins whose predicted PAM sequences correspond to the PAM sequence of interest; (b) evaluating the ability of the one or more CRISPR-Cas proteins to edit the genomic sequence (e.g., in an in vitro gene editing assay, for example as described in Example 1); and (c) selecting a CRISPR-Cas protein from the one or more CRISPR-Cas proteins following the evaluation. The selected CRISPR-Cas protein(s) can be, for example, the CRISPR-Cas protein(s) having the highest editing activity and/or highest fidelity in an in vitro gene editing assay. In some embodiments, step (a) is computer implemented.

In another aspect, the disclosure provides methods for selecting a genomic sequence having a pathogenic mutation for editing with a CRISPR-Cas protein, comprising (a) identifying, in a CRISPR-Cas library of the disclosure, a PAM sequence associated with a pathogenic mutation (e.g., a PAM sequence created by the pathogenic mutation); and (b) selecting a genomic sequence that includes that PAM sequence for editing with a CRISPR-Cas protein. For example, predicted PAM sequences in a library of the disclosure can be aligned with a set of genomic sequences having pathogenic or likely pathogenic mutations, for example mutations in the ClinVar database (Landrum et al., 2018, Nucleic Acids Res. 46: D1062-D1067) to identify genomic sequences that include a PAM sequence present in a library of the disclosure. Genomic sequences that include a sequence that matches exactly to a PAM sequence in the library can be selected for editing. CRISPR-Cas proteins in the library having that PAM sequence can be selected and evaluated for their ability to edit the selected genome sequence. For autosomal dominant diseases, if the PAM sequence is present in the mutant allele but not the wild-type allele, allele specific editing can be achieved.

In another aspect, the disclosure provides methods of designing a guide RNA (gRNA) molecule for a CRISPR-Cas protein in a CRISPR-Cas protein library of the disclosure. The gRNAs can be designed, for example, for a CRISPR-Cas protein selected by a method of the disclosure to edit a genomic sequence of interest, for example, a genomic sequence having a PAM of interest, as described herein. Design of a gRNA can be computer implemented. For example, design of a gRNA can comprise identifying a tracrRNA sequence for a selected CRISPR-Cas protein, e.g., as described in Example 1, and selecting targeting sequence that is complementary to a target genomic sequence and adjacent to the PAM for the selected CRISPR-Cas protein. The designed gRNA can comprise separate crRNA and tracrRNA, or can comprise an sgRNA.

In another aspect, the disclosure provides methods for editing a genomic sequence. Such methods can comprise contacting a cell (e.g., a mammalian cell such as a human cell) with a system comprising a CRISPR-Cas protein selected according to a method described herein and a gRNA for editing the genomic sequence. In some embodiments, the contacting is performed ex vivo. In other embodiments, the contacting is performed in vivo. In some embodiments, the method comprises contacting a cell with a nucleic acid (e.g., an AAV genome) encoding both the CRISPR-Cas protein and the gRNA. In other embodiments, the method comprises contacting a cell with a nucleic acid (e.g., an AAV genome) encoding the CRISPR-Cas protein and a different nucleic acid (e.g., an AAV genome) encoding the gRNA. When the nucleic acid is an AAV genome, the method can comprise contacting the cell with an AAV particle comprising the AAV genome. In other embodiments, the method comprises contacting a cell with a ribonucleoprotein complex comprising the CRISPR-Cas protein and gRNA.

### 7. EXAMPLES

### 7.1. Example 1: Tailored Identification of Cas9 Proteins with Specific PAM Requirements Using a Prediction Computational Pipeline

### 7.1.1. Overview

The identification of the PAM sequences of Cas9 nucleases is crucial for their exploitation as genome editing tools. In this Example, a massively expanded dataset of metagenome and virome assemblies was interrogated for accurate and comprehensive PAM predictions, in order to identify novel Cas9s selected for their PAM requirements. A Cas9 which uses a PAM sequence corresponding to a disease-causing mutation, P23H in the RHO gene, was selectively isolated to illustrate how the described PAM prediction tool can be used to identify and select a CRISPR-Cas protein useful for targeting a PAM of interest, in this case a PAM generated by a disease-causing mutation. The PAM prediction pipeline can be used to generate a CRISPR-Cas (e.g., Cas9) nuclease repertoire responding to any PAM requirement leading towards a natural PAM-free genome editing toolbox.

### 7.1.2. Materials and Methods

### 7.1.2.1. Catalog of Reference and Metagenomic-Assembled Genomes

A catalog of bacterial and archaeal genomic sequences was retrieved from: (i) 257,670 publicly available isolated sequences from the NCBI database (Benson et al., 2013, Nucleic Acids Res. 41: D36-D42 (available as of January 2021), (ii) 771,529 metagenome-assembled genomes (MAGs) from an unpublished study, and (iii) 54, 169 additional MAGs obtained with a validated assembly-based pipeline similarly to Pasolli et al, 2019, Cell 176:649-662.e20. For retrieving these 54, 169 additional MAGs, 8,487 metagenomic samples (Table 1) were assembled using metaSPAdes (Nurk et al., 2017, Genome Res. 27:824-834) if paired-end metagenomes were available, and MEGAHIT (Li et al., 2015, Bioinformatics 31:1674-1676) otherwise. In both cases, default parameters were used. Contigs longer than 1,500 nucleotides were binned into MAGs using MetaBAT2 (Kang et al., 2019, PeerJ 7: e7359).

TABLE 1

| | | | Number | |
|---|---|---|---|---|
| | | | of | Number |
| Study ID | PubMed ID (PMID)/DOI | Environment | samples | of MAGs |
| PaoliL_2021 | 10.1101/2021.03.24.436479 | Ocean | 6261 | 32366 |
| BeresfordJonesBS_2021 | 34971560 | Mice gut | 1043 | 18733 |
| HeroldM_2020 | 33077707 | Wastewater | 787 | 2098 |
| AlbaneseD_2021 | 33741058 | Antarctic desert | 33 | 562 |
| LeechJ_2020 | 33172966 | Fermented food | 70 | 228 |
| LandisEA_2021 | 33496265 | Bakery | 43 | 107 |
| WylensekD_2020 | 33319778 | Pig gut | 38 | 75 |
| KastmanEK_2016 | 27795388 | Dairy | 19 | 56 |
| Unpublished | | Dairy | 35 | 54 |
| LiZ_2018 | 30166168 | Fermented foods | 11 | 49 |
| ArikanM_2020 | 31957879 | Fermented foods | 12 | 35 |
| PatroJN_2016 | 27303722 | Probiotics | 10 | 33 |
| ZhaoCC_2020 | 32247457 | Fermented foods | 5 | 32 |
| ChaconVargasK_2020 | 32934253 | Fermented foods | 8 | 32 |
| PothakosV_2020 | 10.1016/j.crbiot.2020.02.001 | Fermented foods | 10 | 29 |
| PasolliE_2020 | 32451391 | Dairy; Probiotics | 17 | 28 |
| KumarJ_2019 | 31428064 | Fermented foods | 4 | 21 |
| VerceM_2019 | 30918501 | Fermented foods | 6 | 21 |
| YulandiA_2020 | 33308279 | Fermented foods | 2 | 20 |
| EscobarZepedaA_2016 | 27052710 | Dairy | 1 | 18 |
| SulaimanJ_2014 | 25400624 | Brine | 7 | 18 |
| DurulC_2018 | 29803134 | Dairy | 6 | 17 |
| Ferrocinol_2018 | 29196291 | Fermented foods | 11 | 17 |
| DuR_2020 | 32276974 | Alcohol | 3 | 16 |
| LiZ_2019 | 31500701 | Fermented foods | 6 | 13 |
| PorcellatoD_2016 | 10.1016/j.idairyj.2016.05.005 | Dairy | 12 | 13 |
| EinsonEJ_2018 | 30171008 | Fermented foods; | 4 | 12 |
| | | Fruit and Vegetables | | |
| LordanR_2019 | 10.1016/j.jff.2019.01.029 | Dairy | 5 | 11 |
| SalvettiE_2016 | 27445999 | Fruit and Vegetables | 2 | 11 |
| LeonardSR_2016 | 27930729 | Fruit and Vegetables | 7 | 9 |
| DeRoosJ_2020 | 32765478 | Alcohol | 4 | 8 |
| YasirM_2020 | 33233218 | Dairy | 2 | 3 |
| SomervilleV_2019 | 31238873 | Dairy | 1 | 2 |
| CrovadoreJ_2017 | 28572315 | Other | 2 | 2 |

7.1.2.2. Viral Genomes Retrieval from Highly Enriched Viromes

A total of 45,872 viral genomes were metagenomically assembled from 3,044 Human Gut virome datasets as described previously (Karcher et al., 2021, Genome Biol. 22:209). The efficacy of viral enrichment in each virome was evaluated with ViromeQC (Zolfo et al., 2019, Nat. Biotechnol. 37:1408-1412). A total of 255 samples had an enrichment higher than 50× and were retained as highly viral samples. Reads were preprocessed with TrimGalore (version 0.4.4) (Krueger et al., 2021, doi: 10.5281/zenodo.5127899) to remove low quality and short reads (parameters: --stringency 5 --length 75 --quality 20 --max_n 2 --trim-n). Reads aligning to the human genome hg19 were also removed with Bowtie2 (version 2.4.1) (Langmead & Salzberg, 2012, Nat. Methods 9:357-359). High quality reads were assembled into contigs with metaSPAdes (version 3.10.1) (Nurk et al., 2017, Genome Res. 27: 824-834) (k-mer sizes: -k 21,33,55,77,99, 127), or Megahit (version 1.1.1) (Li et al., 2015, Bioinformatics 31 1674-1676).

To reduce non-viral contaminants, contigs that mapped to microbial genomes were removed by using the collection of Metagenomic Assembled Genomes from Pasolli, et al., 2019, Cell 176:649-662.e20. Only contigs that were a) longer than 1500 bp; b) found within the same microbial species-level genome bin in less than 30 metagenomes; and c) found in the unbinned assembled fraction of more than 20 metagenomes, were retained. Contigs from i) the remaining non-highly enriched viromes, and ii) from the human gut metagenomes used in Pasolli, et al., 2019, Cell 176:649-662.e20, and that were similar to a potentially highly enriched viral genome, were also mapped against the unbinned contigs of Pasolli et al. with mash (version 2.0) (Ondov et al., 2016, Genome Biol. 17:132). Contigs with a distance lower than 10% (p-value<=0.05) were retained. Finally, 699 complete viral genomes were selected from RefSeq, release 99 (Brister et al, 2015, Nucleic Acids Res. 43: D571-D577) by selecting genomes that could be found in at least 20 samples within the unbinned contigs of Pasolli, et al., 2019, Cell 176:649-662.e20. All mappings were performed with BLASTN (version 2.6.0) (Altschul et al., 1990, J. Mol. Biol. 215:403-410) identity>80%, aln. len.>1,000 bp). Contigs were clustered at 95% identity with VSEARCH (Rognes et al., 2016, PeerJ 4: e2584) with each cluster needing to contain at least one contig originating from highly enriched viromes.

### 7.1.2.3. PAM Prediction

CRISPRCasTyper (version 1.5.0, default parameters) (Russel et al., 2020, CRISPR J. 3:462-469) was used to identify 131,941 CRISPR-Cas loci. Loci containing Cas9 proteins shorter than 950 aa were excluded from the analysis. The resulting 92, 140 Cas9 proteins were clustered at 100, 99, 98, 97, 96 and 95% identity using UCLUST (version 11.0.667) (Edgar, 2010, Bioinformatics 26:2460-2461) resulting in 27,062, 14,332, 10,475, 8,568, 7,538, and 6,898 clusters respectively.

In total, 613,478 spacers were retrieved from CRISPR arrays and were aligned to 366,233 viral genomes (142,809 from Gut Phage Database (Camarillo-Guerrero et al., 2021, Cell 184:1098-1109.e9), 189,680 from Metagenomic Gut Virus catalog (Nayfach et al., 2021 Nat. Microbiol. 6:960-970) and 45,872 from de novo assembled gut phages from highly enriched viromes using BLASTN (version 2.5.0) (Altschul et al., 1990, J. Mol. Biol. 215:403-410) to identify putative protospacers. Matches with more than 4 mismatches or gaps were filtered out. For each Cas9 clustering level, clusters with less than 10 mapped spacers were discarded, resulting in 7,177 (26.52%), 3,908 (27.27%), 2,779 (26.53%), 2,169 (25.32%), 1,814 (24.06%), and 1,594 (23.11%) clusters. Since the orientation of CRISPR arrays is unknown, both upstream and downstream flanking sequences, up to 30 nucleotides (nt), were retrieved for each putative protospacer. For each Cas9 cluster, protospacer and their flanking sequences, found using the same spacer, were aligned to each other using MUSCLE (version 3.8.31) (Edgar, 2010, Bioinformatics 26:2460-2461) and the alignment was collapsed into a single consensus sequence by taking the most frequent base at each position and discarding columns composed mostly (>50%) of gaps. Spacers were aligned exactly to the consensus sequence to define up- and downstream regions, which were then used to compute nucleotide frequencies and generate sequence logos using Logomaker (version 0.8) (Tareen & Kinney, 2020, Bioinformatics 36:2272-2274).

For each Cas9 cluster, a PAM was considered predicted if there was at least one highly conserved base in only one of the two flanking regions (the PAM can be either upstream or downstream, not both). A highly conserved base was defined as a position in the logo with more information than the maximum between 1 bit and the third quartile plus 1.5 times the interquartile range of the distribution of information in both flanking sequences (i.e. the conserved position is an outlier with at least 1 bit of information). For each clustering level, a PAM was predicted for 6,758 (94.16%), 3,622 (92.68%), 2,546 (91.62%), 1,944 (89.63%), 1,601 (88.26%), and 1,387 (87.01%) clusters with more than 10 mapped spacers.

7.1.2.4. tracrRNA Identification

tracrRNA sequences of the novel Cas9 orthologs were identified computationally, searching for sequences starting with a putative anti-repeat and ending with a Rho-independent transcription terminator (RIT). Putative anti-repeats were identified aligning CRISPR repeats to sequences flanking the CRISPR-Cas locus (up to 1,000 nt) using BLASTN (version 2.5.0) (Altschul et al., 1990, J. Mol. Biol. 215:403-410) and RITs were predicted using RNIE (Gardner et al., 2011, Nucleic Acids Res. 39:5845-5852) 7.1.2.5. In vitro PAM determination

In vitro PAM evaluation of the novel Cas9 orthologs was performed according to the protocol from Karvelis et al., 2019, Methods in Enzymology 616:219-240. In brief: for each Cas9 ortholog the human codon optimized version of its coding sequences was ordered as a synthetic construct (Genscript) and cloned into an expression vector for in vitro transcription and translation (IVT) (pT7-N-His-GST-Thermo Fisher Scientific). Reactions were performed according to the manufacturer's protocol (1-Step Human High-Yield Mini IVT Kit-Thermo Fisher Scientific). The Cas9-guideRNA RNP complex was assembled by combining 20 μL of the supernatant containing soluble Cas9 protein with 1 μL of RiboLock RNase Inhibitor (Thermo Fisher Scientific) and 2 μg of guide RNA. The Cas9-guideRNA complex obtained was used to digest 1 μg of a plasmid (p11-lacY-wtx backbone-Addgene #69056) containing an 8-nucleotide randomized PAM sequence flanking the gRNA target. Digestion reactions were incubated for 1 hour at 37° C.

A double-stranded DNA adapter was then ligated to the DNA ends generated by the targeted Cas9 cleavage and the final ligation product was purified using a GeneJet PCR Purification Kit (Thermo Fisher Scientific).

One round of a two-step PCR (Phusion HF DNA polymerase-Thermo Fisher Scientific) was performed to enrich the sequences that were cut using a set of forward primers annealing on the adapter and a reverse primer designed on the plasmid backbone downstream of the PAM (Table 2). A second round of PCR was performed to attach the Illumina indexes and adapters. PCR products were purified using Agencourt AMPure beads in a 1:0.8 ratio.

TABLE 2
Sequences of the primers used for NGS library
preparation in the in vitro PAM assay

| | | SEQ |
|---|---|---|
| Primer | | ID |
| name | Sequence (5′ -> 3′) | NO |
| F4a | TCGTCGGCAGCGTCAGATGTGTATAAGAGACAGCTG | 1 |
| | CTGAACCGCTCTTCCGATC | |
| F4b | TCGTCGGCAGCGTCAGATGTGTATAAGAGACAGTAA | 2 |
| | GACTGCTGAACCGCTCTTCCGATC | |
| F4c | TCGTCGGCAGCGTCAGATGTGTATAAGAGACAGGCT | 3 |
| | AGACCTAATGTGATCTGCTGAACCGCTCTTCCGATC | |
| R3 | GTCTCGTGGGCTCGGAGATGTGTATAAGAGACAGTC | 4 |
| | TGCGTTCTGATTTAATCTGTATCAGGC | |

The generated library was analyzed with a 71-bp single read sequencing, using a flow cell v2 micro, on an Illumina MiSeq sequencer.

PAM sequences were extracted from Illumina MiSeq reads and used to generate PAM sequence logos. PAM heatmaps (Walton et al., 2020, Science 368:290-296) were used to display PAM enrichment, computed dividing the frequency of PAM sequences in the cleaved library by the frequency of the same sequences in a control uncleaved library.

### 7.1.2.6. PAM Comparison and Hierarchical Clustering

Differences between PAM sequences were quantified using the Jensen-Shannon distance (defined as the square root of the Jensen-Shannon divergence) (Nettling et al., 2015, BMC Bioinformatics 16:387). PAM predictions resulting from the 98% identity Cas9 clustering showed the lowest median distance from the in vitro determined PAMs of Cas9 orthologs characterized by Gasiunas et al. (Gasiunas et al., 2020, Nat. Commun. 11:5512) and were therefore chosen for subsequent analyses. An all-to-all PAM prediction distance matrix was computed and hierarchical clustering was performed to generate PAM clusters, using usearch (version 11.0.667, parameters-cluster_aggd-id 0.6-linkage avg) (Edgar, 2010, Bioinformatics 26:2460-2461). Consensus PAMs for each cluster were generated using the protospacer flanking sequences of each cluster member.

7.1.2.7. PAM Clusters Association with Cas9 Phylogenetic Tree

Cas9 proteins with a predicted PAM (98% identity clustering) were aligned using mafft (version 7.490, with parameters --maxiterate 10) (Katoh & Standley, 2013, Mol. Biol. Evol. 30:772-780) and a phylogenetic tree was built using FastTree (version 2.1.11, with parameters -spr 4 -mlacc 2 -slownni) (Price et al., 2010, PLOS ONE 5: e9490). Cas9 clades were defined using TreeCluster (version 1.0.3, with parameters -m max_clade) (Balaban et al., 2019 PLOS ONE 14: e0221068) and a range of thresholds (0.3 to 4). Associations between PAM clusters and Cas9 clades were assessed using Fisher's exact test, computing p-values by Monte Carlo simulation with 100,000 replicates and a 0.001 significance level.

### 7.1.2.8. Identification of PAM-Matching Mutations in ClinVar

Mutations in the ClinVar database (accessed Mar. 6, 2022) (Landrum et al., 2018, Nucleic Acids Res. 46: D1062-D1067) were filtered to select single nucleotide variants and short InDels (10 or less nucleotides) annotated as pathogenic or likely pathogenic and associated with pathologies with known mode of inheritance, for a total of 89,751 mutations. To compute the fraction of mutations that can be targeted by at least a Cas9 in the databank with allelic discrimination, predicted PAM sequences resulting from the 98% identity Cas9 clustering were aligned exactly to wild-type and mutated alleles.

### 7.1.2.9. Cell Culture and InDels Analysis

HEK293T/17 cells obtained from ATCC were cultured in DMEM supplemented with 10% fetal bovine serum, 2 mM L-Glutamine, 100 U/ml Penicillin and 100 μg/ml streptomycin (Life Technologies) and incubated at 37° C. and 5% CO₂ in a humidified atmosphere. Cells tested mycoplasma negative (PlasmoTest, Invivogen). For InDels analyses cells were seeded in 24-well plate and transfected after 24 hours with 1000 ng pX-PrCas9-sgRNA-RHO-P23H, 50 ng pCMV-TO-RHO-P23H or pCMV-TO-RHO-WT and 50 ng pEGFP-IRES-Puro using TransIT-LT1 transfection reagent (Mirus Bio) according to manufacturer's instructions. 48 hours post-transfection cells were pool-selected with 1 μg/ml Puromycin and collected after 72 hours. Genomic DNA was obtained from cell pellets using the QuickExtract DNA extraction solution (Lucigen) according to the manufacturer's instructions. The RHO P23 locus was amplified using the HOT FIREPol Multiplex Mix (Solis Biodyne) with primers RHO-TO-F (CAGTGATAGAGATCTCCCTATC) (SEQ ID NO:5) and RHO-int1-R (GAGATAGATGCGGGCTTCCA) (SEQ ID NO:6). PCR amplicons were purified using CleanNGS beads (CleanNA) and Sanger sequenced (Microsynth) using RHO-TO-F primer. Indel levels were evaluated using TIDE (Brinkman et al., 2014, Nucleic Acids Res. 42: e168-e168).

### 7.1.2.10. Plasmids

A pX330-derived plasmid was used to express the Cas9 orthologs in mammalian cells. Briefly, pX330 (Addgene) was modified by substituting SpCas9 and its sgRNA scaffold with the human codon optimized coding sequence of the variant of interest and its sgRNA scaffold. The Cas9 variants coding sequences, modified, as described before, by the addition of an SV5 tag at the N-terminus and two nuclear localization signals (1 at the N-term and 1 at the C-term) and human codon-optimized, as well as the sgRNA scaffolds, were obtained as synthetic fragments from either Genscript or Genewiz. Spacer sequences were cloned into the pX-Cas9 plasmids as annealed DNA oligonucleotides containing a variable 20 or 24-nt spacer sequence using a double Bsal site present in the plasmid. The list of spacers sequences used in the EGFP disruption assay and in the evaluation of editing activity against the RHO P23H mutation is reported in Table 3.

TABLE 3
Sequences of the oligonucleotides used for spacers cloning in the expression plasmids

| | | SEQ | | SEQ |
|---|---|---|---|---|
| Plasmid | Oligonucleotide 1 (5′-3′) | ID NO | Oligonucleotide 2 (5′-3′) | ID NO |
| pX-SpCas9- | CACCGGGCACGGGCAGC | 7 | GAACCCGGCAAGCTGC | 8 |
| sgRNA- GFPB | TTGCCGG | | CCGTGCCC | |
| pX-PrCas9- | CACCGCCCGAAGGCTAC | 9 | GAACCGCTCCTGGACGT | 10 |
| sgRNA-GFP | GTCCAGGAGCG | | AGCCTTCGGGC | |
| pX-PrCas9- | CACCGCAGCCAGGTAGT | 11 | GAACAGTACCCACAGTA | 12 |
| sgRNA- RHO- | ACTGTGGGTACT | | CTACCTGGCTGC | |
| P23H | | | | |

pCMV-TO-RHO-WT plasmid was obtained by cloning the human rhodopsin (RHO) gene into the pCDNA5/TO plasmid (Addgene). The hRHO gene was PCR-amplified using the primers RHO_gene_F (attaggatccAGAGTCATCCAGCTGGAGCCC) (SEQ ID NO:13) and RHO_gene_R (taatctcgagTGGGGTTTTTCCCATTCCCAGG) (SEQ ID NO:14) from genomic DNA extracted from HEK293T/17 cells using the Phusion high fidelity DNA Polymerase (ThermoFisher Scientific). The P23H mutation was further inserted by site-directed mutagenesis using primers mut-P23H-F (GTGTGGTACGCAGCCaCTTCGAGTACCCACAG) (SEQ ID NO:15) and mut-P23H-R (CTGTGGGTACTCGAAGGGCTGCGTACCACAC) (SEQ ID NO: 16) to generate pCMV-TO-RHO-P23H plasmid. All oligonucleotides were purchased from Eurofins Genomics.

### 7.1.3. Results and Discussion

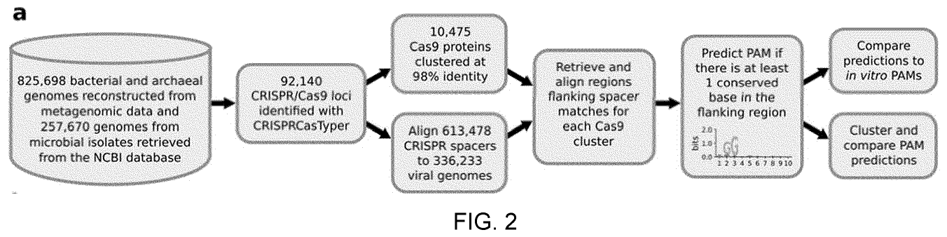

Interrogation of massive metagenomic datasets combined with a newly developed computational method allowed for the identification of a vast number of unreported CRISPR-Cas loci and their respective PAM requirements (pipeline schematized in FIG. 2). A search focused on Type II systems was performed since their simplicity facilitates their biotechnological exploitation (Makarova et al., 2020, Nat. Rev. Microbiol. 18:67-83). From 825,698 bacterial and archaeal genomes reconstructed via metagenomic assembly of human, host-associated, and non-host associated environmental microbiomes (see Section 7.1.2) and 257,670 genomes from microbial isolates retrieved from the NCBI database, 92,140 CRISPR-Cas9 loci were identified. Cas9 proteins were clustered at multiple sequence identity levels (from 100-95%) and the PAM prediction analysis was performed for each level to identify the best approach for PAM prediction accuracy (clustering at 98% nucleotide identity, see Section 7.1.2). To identify protospacer flanking sequences, 613,478 unique spacers were aligned to phage genomes of the human microbiome: 142,809 from the Gut Phage Database (Camarillo-Guerrero et al., 2021, Cell 184:1098-1109.e9), 189,680 from the Metagenomic Gut Viral catalog (Nayfach et al., 2021 Nat. Microbiol. 6:960-970) and 45,872 from de novo assembled gut phages from highly enriched viromes as profiled via ViromeQC (Zolfo et al., 2019, Nat. Biotechnol. 37:1408-1412) (see Section 7.1.2). Only full-length, near-perfect matches were retained (at most 4 nucleotide variations), resulting in a total of 39, 109,402 putative protospacers. Cas9 clusters with less than 10 mapped spacers were discarded to retain only highly reliable PAM predictions. Upstream and downstream sequences flanking the matches, up to 30 nt, were retrieved. For each Cas9 cluster, sequences flanking the same spacer were realigned and the multiple sequence alignment was collapsed into a single consensus flanking sequence, to normalize the match counts. Nucleotide frequencies in the consensus flanking sequences were computed and represented as sequence logos. A PAM was predicted for a Cas9 cluster if there was at least one conserved position in either the upstream or the downstream flanking sequence. In total, for the 98% identity clustering, we obtained PAM predictions for 2,546 out of 2,779 Cas9 clusters (representing a total of 61,095 Cas9 sequences) with more than 10 mapped spacers (91.6%).

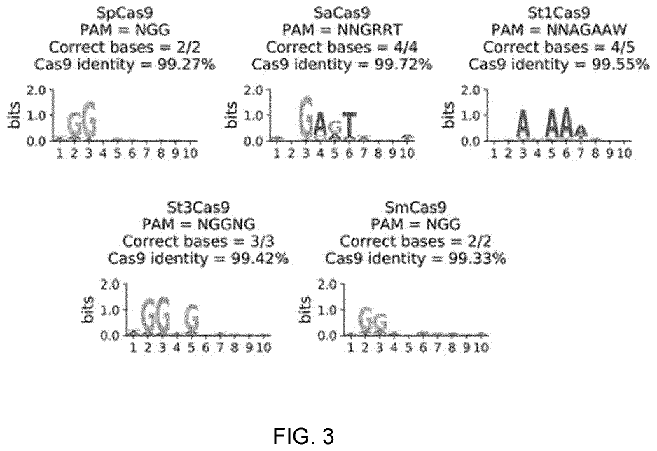

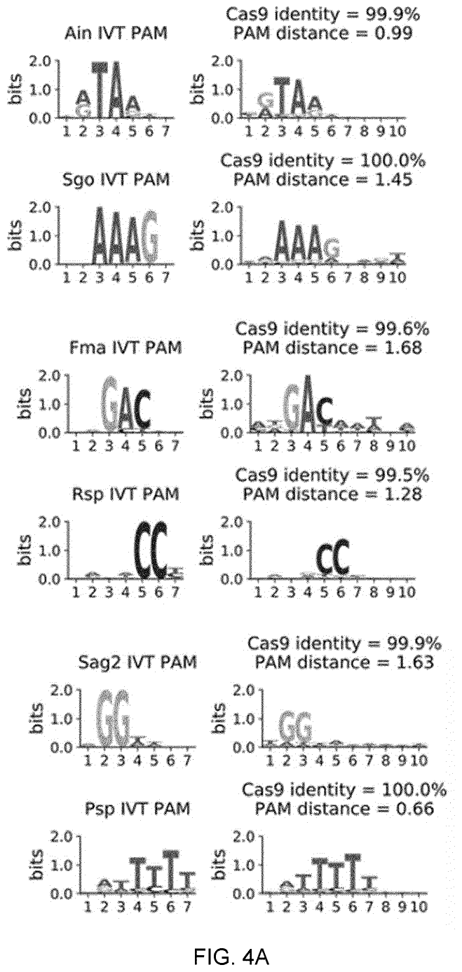

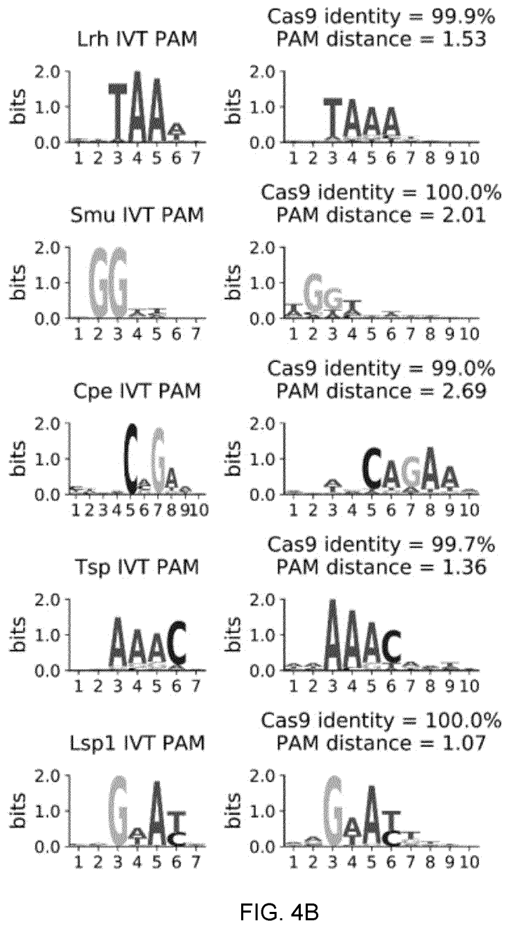

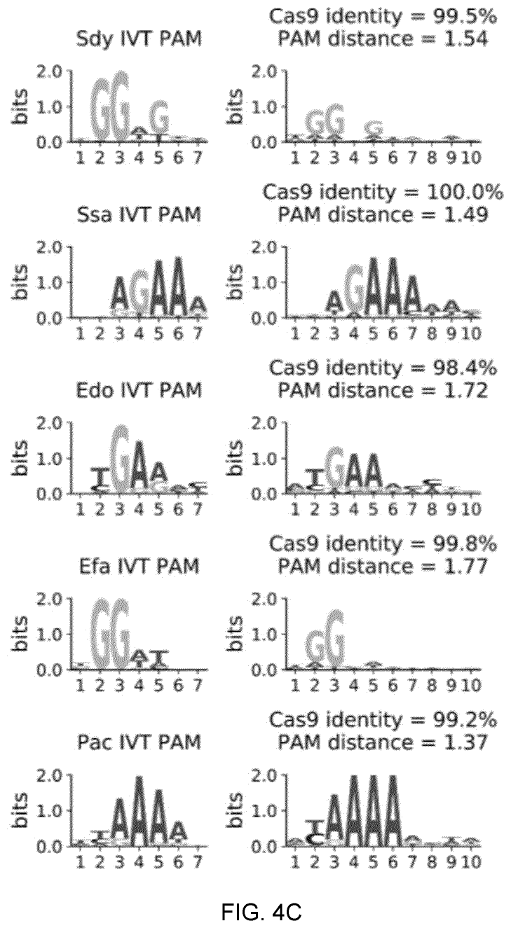

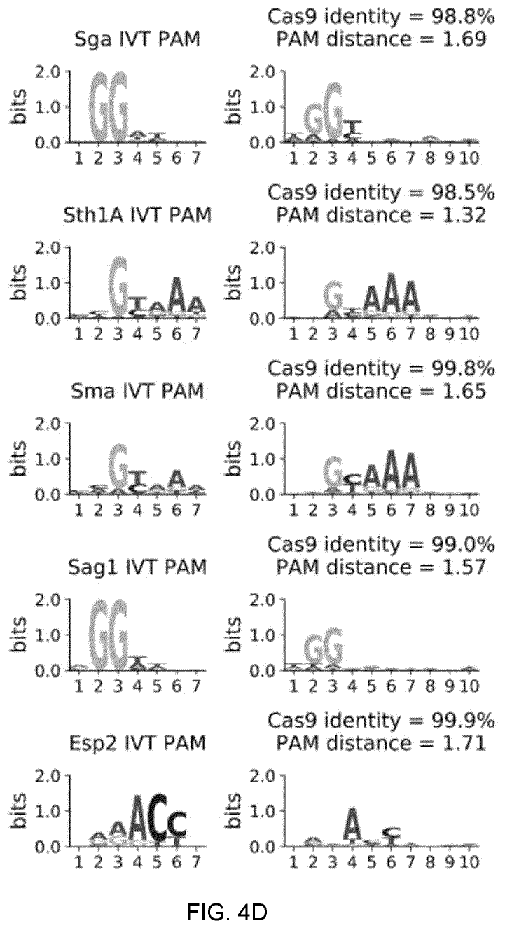

To validate the approach and predictions, gene sequences coding for proteins with high sequence identity (>98%) to previously characterized Cas9s were searched for in the dataset: SpCas9 (Jinek et al., 2021, Science 337:816-821), SaCas9 (Ran et al., 2015, Nature 520:186-191), St1Cas9 (Garneau et al., 2010, Nature 468:67-71), St3Cas9 (Horvath et al., 2008, J. Bacteriol. 190:1401-1412) and SmCas9 (Shields et al., 2020, PLOS Pathog. 16: e1008344). For these Cas9s we obtained sequence predictions corresponding to the described PAMs (FIG. 3). The method was further validated by cross checking the PAM predictions obtained with the pipeline with the sequences experimentally identified and recently reported and characterized in Gasiunas et al., 2020, Nat. Commun. 11:5512. Of the 79 Cas9s reported, 21 could be used for the evaluation here as they have a close ortholog in the dataset (>98% identity), and for them the accuracy of the prediction strategy was confirmed by obtaining PAM logos with high identity (assessed by Jensen-Shannon distance on nucleotide frequencies, see Section 7.1.2) with the sequences determined experimentally (FIGS. 4A-4D). Overall, 85% of PAM predictions generated by the method of this Example were correct and the remaining 15% were partial predictions with at least one base correctly identified. The method of this Example exhibited a much higher prediction accuracy compared to Spacer2PAM (Rybnicky et al., 2022, Nucleic Acids Res. 50:3523-3534), the best PAM prediction method reported previously (45% correct predictions, 55% partial predictions). Based on the median PAM distance between these 21 PAMs and the predictions of the method of this Example, it was determined that the Cas9 clustering at 98% identity generated slightly better PAM predictions than the other clustering levels. This clustering was chosen for subsequent analyses.

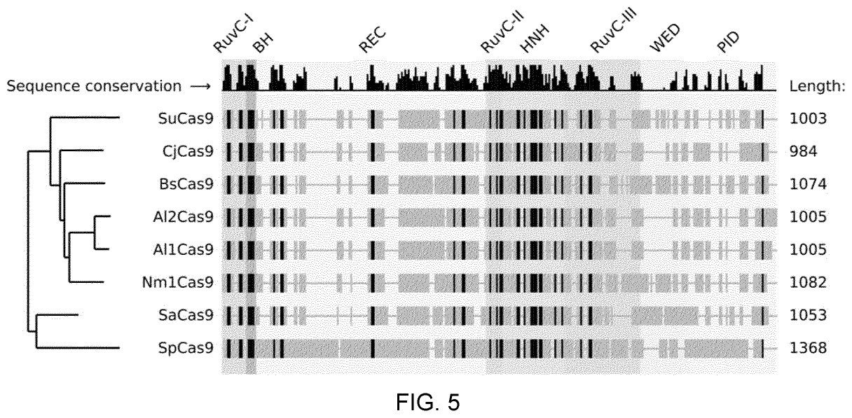

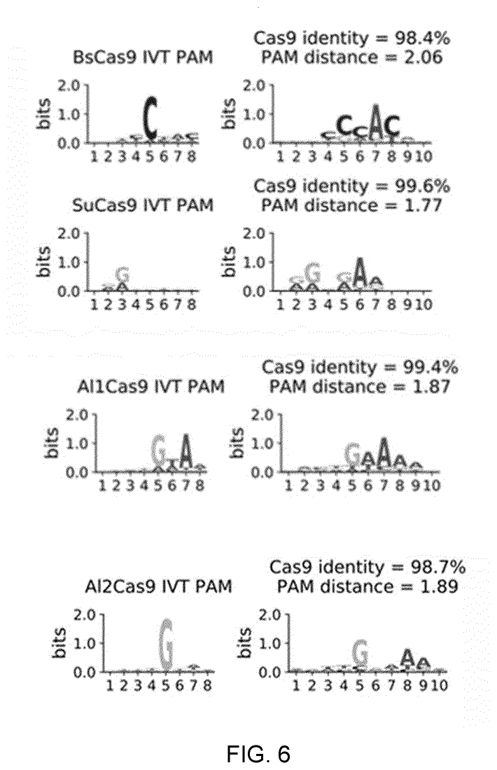

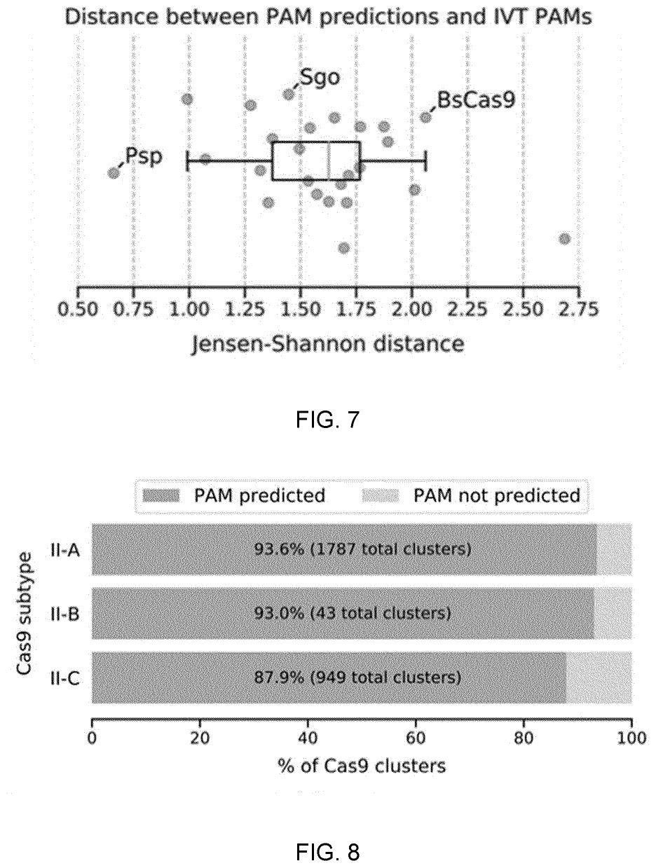

To further evaluate the reliability and potential of the PAM prediction pipeline in expanding the Cas9 toolbox from the databank, Cas9 candidates were searched for using parameters favoring the identification of functionally active enzymes (with preserved domain structures and located in complete CRISPR-Cas loci) and with reduced molecular size (<1,100 amino acids), thus potentially more convenient for genome editing applications. Four Cas9 never described before from poorly characterized species were identified (FIG. 5). Their PAM logos were predicted and subsequently validated through an in vitro assay (Karvelis et al., 2019, Methods in Enzymology 616:219-240). Results demonstrated a very close identity between in silico and in vitro results as indicated by the small distance (less than 2 bits for 3 out of 4 Cas9 variants) between predicted and in vitro determined PAMs (FIG. 6 and FIG. 7), thus further confirming the accuracy and the potential of this PAM prediction pipeline. Overall, the method of this Example allowed PAM prediction for the vast majority of Cas9 proteins identified in the databank with 10 or more mapped spacers, across all Cas9 subtypes (93.6% for A, 93.0% for B and 87.9% for C) (FIG. 8).

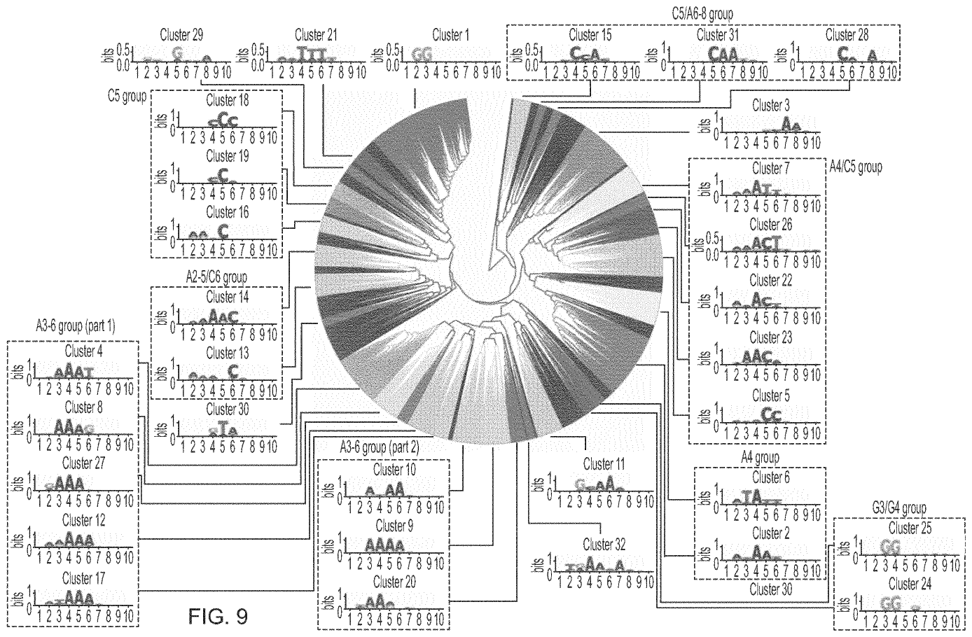

The PAM predictor method was then applied to the metagenomically extended set of 2,546 Cas9 protein families (98% identity clustering) to identify all PAM requirements and explore whether specific PAM clusters may exist. Hierarchical clustering on pairwise distance of the predicted PAMs retrieved 32 clusters with at least 20 members (see Section 7.1.2). For each PAM cluster, a consensus PAM was generated (FIG. 9). Interestingly, the most prevalent PAM sequences represented only a small fraction of all possible PAMs. Therefore, even though the PAM variability is high for Type II Cas9 (Vink et al., 2021, Genome Biol. 22:281), only definite combinations of nucleotides were identified.

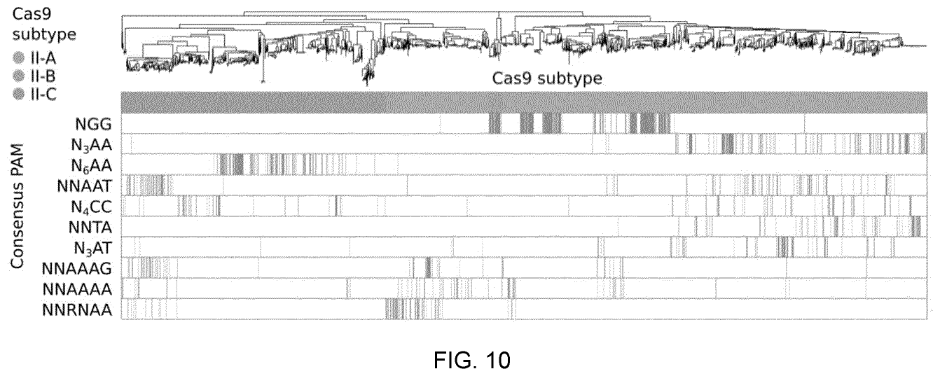

It was then evaluated whether there might be an association between the PAM clusters identified in FIG. 9 and specific Cas9 subtypes. After generating a phylogenetic tree of the identified Cas9 proteins, it was found that almost every PAM cluster was associated with specific clades of Cas9 proteins (FIG. 10), thus suggesting a non-random organization of PAM recognition sequences. For instance, the most abundant PAM cluster (NGG) was found in a specific branch of type II-A and in almost all type II-B Cas9s.

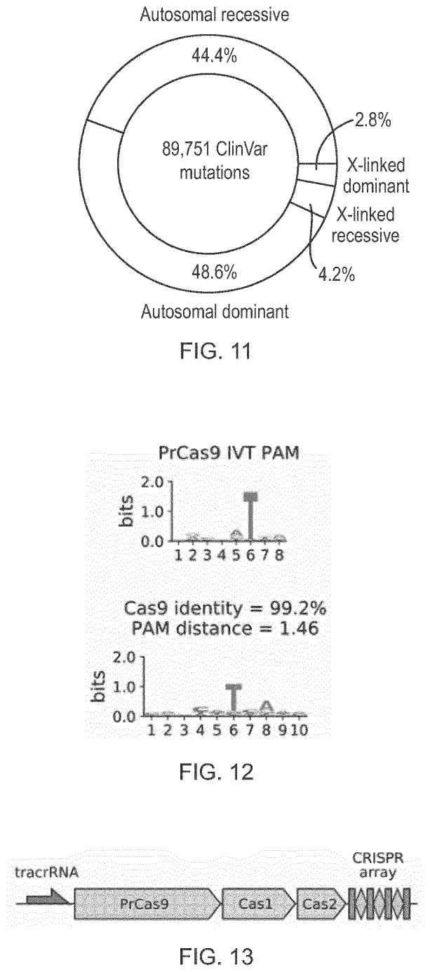

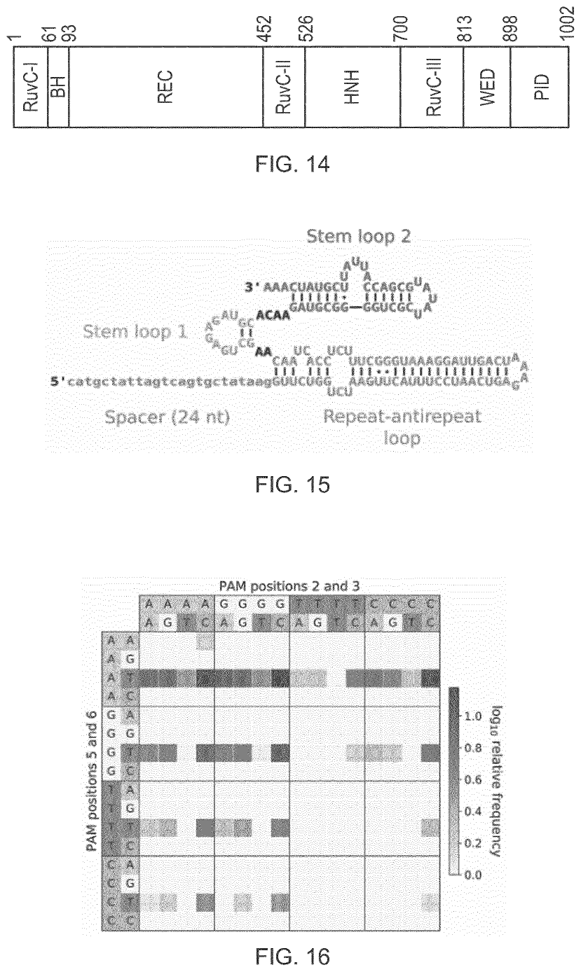

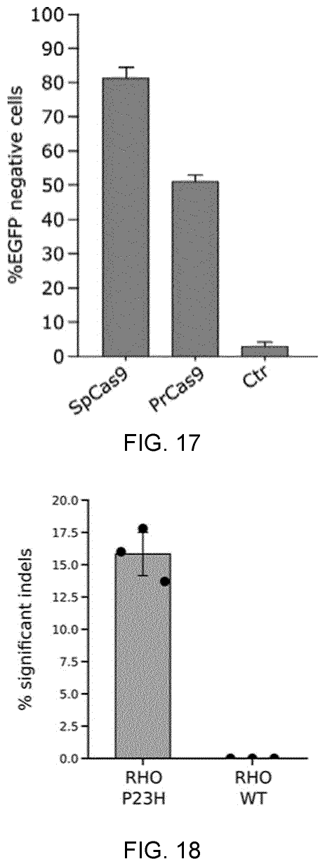

A promising and simple therapeutic application of the CRISPR-Cas technology is the knock-out of mutations causing autosomal dominant genetic diseases. Nonetheless, allelic discrimination is hardly obtained through CRISPR-Cas due to various grades of sgRNA mismatch tolerance by Cas9. Conversely, since PAM sequences are stringent requirements for Cas9 activity, targeting mutated alleles generating novel PAMs would allow a specific target separation between the mutated and the wild-type alleles. Consequently, a paramount application of the PAM prediction pipeline described in this Example is the identification of novel Cas9s recognizing PAM sequences generated by pathogenic mutations to offer specific targeting options for the mutated allele with a highly secured allelic discrimination. By interrogating the ClinVar database (Landrum et al., 2018, Nucleic Acids Res. 46: D1062-D1067) for mutations corresponding to PAMs associated with Cas9s from the metagenomic analysis, it was found that a large fraction of pathogenic mutations (98.6% of 89,751 substitutions and small indels with known mode of inheritance) were included in at least one of the identified PAMs, thus providing allelic discrimination, with 48.7% of them being autosomal dominant alterations (FIG. 11). As a proof of concept for the potential of the PAM prediction method described in this Example, a specific dominant-negative mutation, the P23H mutation in rhodopsin gene (Dryja et al., 1991, Proc. Natl. Acad. Sci. U.S.A 88:9370-9374), was chosen for further study. The P23H mutation is the most common mutation causing RHO dependent retinitis pigmentosa (Hamel, 2006, Orphanet J. Rare Dis. 1:40). PrCas9, a Cas9 found in an unclassified Proteobacteria species, which has a predicted PAM N5T corresponding to a P23H mutation in RHO (CGAAGT, wild-type sequence CGAAGG) was identified and its PAM preference was validated in vitro (PAM NRVNRT, FIG. 12-FIG. 16). PrCas9 editing activity was first evaluated in an EGFP disruption assay to verify its activity in mammalian cells generating near 50% EGFP disrupted cells (FIG. 17) and then towards RHO wild-type or carrying the P23H mutation. Up to 15.8% InDels at the RHO P23H locus and the complete absence of indels in the wt sequence were obtained, thus demonstrating the efficacy of the selected Cas9 in targeting the RHO specific mutation in mammalian cells (FIG. 18).

The PrCas9 amino acid sequence is set forth below:

(SEQ ID NO: 17)
MKMQDSVSKMKYRLGIDLGTTSLGWAMLRLDEQNEPYAVIRAGVRIFNNG
RDPKTEASLAVARRLARQQRRTRDRKIRRKERLIGELVDMGFFPKDPVKR
RQLASLDPFKLRTEALDRALSPEEFARAIFHLARRRGFKSNRKTDSGDTE
SSKMKEAIKRTLNELQNKGFRTVGEWLNMRHQQRLGTRSRIKNVPTGSGK
QTTAYDFYLNRFMIEYEFDRIWEKQSQMNPGLFTNERKAILKDIIFYQRP
LRPVEPGRCTFMPDNPRAPLALPQQQDFRIYQEVNNLRKIDPTSLLEVNL
TLPERDRIVELLQRKPALTFDAVRKALCFNGTFNLEGENRSELKGNLTNC
ALAKKKLFGESWYSFDAHKRFEIVEHLLQEESEENLVSWLQKECNLSEEY
AKNVASVRLPAGYGALCQEALDLILPYLKAEVITYDKAVQKAGMNHSELT
LAQETGEILPELPYYGQYLKRHVGFGTGKPEDSAEKRYGKIPNPTVHIAL
NQLRTVVNALIRRYGKPTQIVIELARELKQNKKAKDQYRIEMNHNQNRNE
RIRADISMILGINPENVKRKDIEKQILWEELNLKDATARCCPYSGKQISA
EMLFTDEVEIDHILPFSRTLDDSKNNKVVCIREANRIKGNRTPWEARKDF
EKRGWSVEAMTARAQAMPKAKRFRFAEDGYKVWLKDFDGFEARALTDTQY
MSRVAREYLQLICPGQTWSVPGQLTGMLRRFLGLNDILGVNGEKNRDDHR
HHAVDACVIALTDRSMLQRISTASARAENKHLTRLLESFPAPWATFYEHV
TRAVKSICVSHKPEHAYQGAMNEQTAYGLRPDGYVKYRQNGKVEHKKLNV
IPQVSVKGTWRHGLNSDGSLKAYKGLKGGSNFCIEIVMGEGGRWEGDVIT
TYEAYQIVRAKGEAALYGSVSRSGKPLVMRLMQKDIVEMTLADGRCKMLL
YIITQNKQMFFYRIENAGGGREDVSKRPGSLQKALAKKIIVSPIGDFRKE
KL

In conclusion, by interrogating an extended microbiome databank with an accurate computational pipeline, a large variety of new Cas9 nucleases was identified accompanied by their PAM requirements. This analysis revealed that PAM sequences follow defined nucleotide patterns which are associated with specific Cas9 subtypes and overlap with 98.6% of the pathogenic mutations reported in ClinVar (Landrum et al., 2018, Nucleic Acids Res. 46: D1062-D1067). The precise PAM prediction driven by a specific sequence-mutation query allows for the identification of tailored Cas9s, such as PrCas9 targeting the P23H RHO mutation. This approach opens to the expansion of the genome editing toolbox with mutation-tailored nucleases and supports the strategy of an application-specific search for suitable natural prokaryotic genome editing tools requiring minimal or no engineering.

### 8. SPECIFIC EMBODIMENTS, CITATION OF REFERENCES

While various specific embodiments have been illustrated and described, it will be appreciated that various changes can be made without departing from the spirit and scope of the disclosure(s). The present disclosure is exemplified by the numbered embodiments set forth below.
1. A method for predicting a protospacer adjacent motif (PAM) sequence recognized by one or more CRISPR-Cas proteins in a CRISPR-Cas protein cluster, comprising:
   a) mapping spacer sequences of one or more CRISPR arrays corresponding to one or more CRISPR-Cas proteins of the CRISPR-Cas protein cluster to a set of viral genome sequences to identify putative protospacer sequences in the viral genomes and their flanking upstream and downstream sequences in the viral genomes, wherein:
      i) mapping the spacer sequences to the viral genomes comprises aligning the spacer sequences to the viral genome sequences; and
      ii) the CRISPR-Cas protein cluster comprises a set of CRISPR-Cas protein sequences clustered by sequence identity;
   b) for each spacer sequence mapped to one or more viral genome sequences, aligning the putative protospacer sequences and their flanking upstream and downstream viral genome sequences to generate a set of aligned putative protospacer and flanking sequences for each mapped spacer sequence; and
   c) predicting a PAM sequence recognized by one or more CRISPR-Cas proteins in the cluster from the sets of aligned putative protospacer and flanking sequences.
2. The method of embodiment 1, which is computer implemented.
3. A computer-implemented method for predicting a protospacer adjacent motif (PAM) sequence recognized by one or more CRISPR-Cas proteins in a CRISPR-Cas protein cluster, the method comprising executing, in a computer system having one or more processors coupled to a memory storing one or more computer readable instructions for execution by the one or more processors, the one or more computer readable instructions comprising instructions for:
   a) mapping spacer sequences of one or more CRISPR arrays corresponding to one or more CRISPR-Cas proteins of the CRISPR-Cas protein cluster to a set of viral genome sequences to identify putative protospacer sequences in the viral genomes and their flanking upstream and downstream sequences in the viral genomes, wherein:
      i) mapping the spacer sequences to the viral genomes comprises aligning the spacer sequences to the viral genome sequences; and
      ii) the CRISPR-Cas protein cluster comprises a set of CRISPR-Cas protein sequences clustered by sequence identity;
   b) for each spacer sequence mapped to putative protospacer sequences, aligning the protospacer sequences and their flanking upstream and downstream viral genome sequences to generate a set of aligned putative protospacer and flanking sequences for each mapped spacer sequence; and
   c) predicting a PAM sequence recognized by one or more CRISPR-Cas proteins in the cluster from the sets of aligned putative protospacer and flanking sequences.
4. The method of any one of embodiments 1 to 3, wherein the CRISPR-Cas protein cluster comprises at least 1 CRISPR-Cas protein sequence.
5. The method of any one of embodiments 1 to 3, wherein the CRISPR-Cas protein cluster comprises at least 10 CRISPR-Cas protein sequences.
6. The method of any one of embodiments 1 to 3, wherein the CRISPR-Cas protein cluster comprises at least 100 CRISPR-Cas protein sequences.
7. The method of any one of embodiments 1 to 3, wherein the CRISPR-Cas protein cluster comprises at least 1000 CRISPR-Cas protein sequences
8 The method of any one of embodiments 1 to 7, wherein the CRISPR-Cas protein cluster comprises up to 1000 CRISPR-Cas protein sequences.
9 The method of any one of embodiments 1 to 7, wherein the CRISPR-Cas protein cluster comprises up to 2000 CRISPR-Cas protein sequences.
10. The method of any one of embodiments 1 to 7, wherein the CRISPR-Cas protein cluster comprises up to 5000 CRISPR-Cas protein sequences.
11. The method of any one of embodiments 1 to 7, wherein the CRISPR-Cas protein cluster comprises up to 10,000 CRISPR-Cas protein sequences.
12. The method of any one of embodiments 1 to 11, wherein the CRISPR-Cas protein sequences in the cluster are at least 150 amino acids in length.
13. The method of embodiment 12, wherein the CRISPR-Cas protein sequences in the cluster are at least 200 amino acids in length
14. The method of embodiment 12, wherein the CRISPR-Cas protein sequences in the cluster are at least 500 amino acids in length
15. The method of embodiment 12, wherein the CRISPR-Cas protein sequences in the cluster are at least 700 amino acids in length
16. The method of embodiment 12, wherein the CRISPR-Cas protein sequences in the cluster are at least 900 amino acids in length
17. The method of embodiment 12, wherein the CRISPR-Cas protein sequences in the cluster are at least 950 amino acids in length.
18. The method of embodiment 12, wherein the CRISPR-Cas protein sequences in the cluster are at least 1000 amino acids in length.
19. The method of embodiment 12, wherein the CRISPR-Cas protein sequences in the cluster are at least 1100 amino acids in length.
20. The method of embodiment 12, wherein the CRISPR-Cas protein sequences in the cluster are at least 1200 amino acids in length.
21. The method of embodiment 12, wherein the CRISPR-Cas protein sequences in the cluster are at least 1300 amino acids in length.
22. The method of any one of embodiments 1 to 21, wherein the CRISPR-Cas protein sequences in the cluster are less than 2200 amino acids in length.
23. The method of any one of embodiments 1 to 21, wherein the CRISPR-Cas protein sequences in the cluster are less than 2100 amino acids in length.
24. The method of any one of embodiments 1 to 21, wherein the CRISPR-Cas protein sequences in the cluster are less than 2000 amino acids in length.
25. The method of any one of embodiments 1 to 21, wherein the CRISPR-Cas protein sequences in the cluster are less than 1800 amino acids in length.
26. The method of any one of embodiments 1 to 21, wherein the CRISPR-Cas protein sequences in the cluster are less than 1600 amino acids in length
27. The method of any one of embodiments 1 to 21, wherein the CRISPR-Cas protein sequences in the cluster are less than 1500 amino acids in length
28. The method of any one of embodiments 1 to 21, wherein the CRISPR-Cas protein sequences in the cluster are less than 1400 amino acids in length
29. The method of any one of embodiments 1 to 20, wherein the CRISPR-Cas protein sequences in the cluster are less than 1300 amino acids in length
30. The method of any one of embodiments 1 to 19, wherein the CRISPR-Cas protein sequences in the cluster are less than 1200 amino acids in length.
31. The method of any one of embodiments 1 to 18, wherein the CRISPR-Cas protein sequences in the cluster are less than 1100 amino acids in length.
32. The method of any one of embodiments 1 to 31, wherein each CRISPR-Cas protein sequence in the cluster has at least 95% sequence identity to a centroid sequence.
33. The method of embodiment 32, wherein each CRISPR-Cas protein sequence in the cluster has at least 96% sequence identity to a centroid sequence.
34. The method of embodiment 32, wherein each CRISPR-Cas protein sequence in the cluster has at least 97% sequence identity to a centroid sequence.
35. The method of embodiment 32, wherein each CRISPR-Cas protein sequence in the cluster has at least 98% sequence identity to a centroid sequence.
36. The method of embodiment 32, wherein each CRISPR-Cas protein sequence in the cluster has at least 99% sequence identity to a centroid sequence.
37 The method of embodiment 32, wherein each CRISPR-Cas protein sequence in the cluster has 100% sequence identity to a centroid sequence.
38. The method of any one of embodiments 1 to 37, further comprising a step of clustering an initial set of CRISPR-Cas protein sequences to generate the CRISPR-Cas protein cluster.
39. The method of embodiment 38, wherein the initial set of CRISPR-Cas protein sequences comprises at least 10,000 CRISPR-Cas protein sequences.
40. The method of embodiment 38, wherein the initial set of CRISPR-Cas protein sequences comprises at least 50,000 CRISPR-Cas protein sequences.
41. The method of embodiment 38, wherein the initial set of CRISPR-Cas protein sequences comprises at least 75,000 CRISPR-Cas protein sequences.
42. The method of embodiment 38, wherein the initial set of CRISPR-Cas protein sequences comprises at least 90,000 CRISPR-Cas protein sequences.
43. The method of any one of embodiments 38 to 42, wherein the initial set of CRISPR-Cas protein sequences comprises up to 1 million CRISPR-Cas protein sequences.
44. The method of any one of embodiments 38 to 42, wherein the initial set of CRISPR-Cas protein sequences comprises up to 750,000 CRISPR-Cas protein sequences.
45. The method of any one of embodiments 38 to 42, wherein the initial set of CRISPR-Cas protein sequences comprises up to 500,000 CRISPR-Cas protein sequences.
46. The method of any one of embodiments 38 to 42, wherein the initial set of CRISPR-Cas protein sequences comprises up to 200,000 CRISPR-Cas protein sequences.
47. The method of any one of embodiments 38 to 42, wherein the initial set of CRISPR-Cas protein sequences comprises up to 100,000 CRISPR-Cas protein sequences.
48. The method of any one of embodiments 38 to 47, wherein the step of clustering the initial set of CRISPR-Cas protein sequences comprises clustering the initial set of CRISPR-Cas protein sequences by a sequence-based clustering algorithm.
49. The method of embodiment 48, wherein the sequence-based clustering algorithm is UCLUST.
50. The method of embodiment 48, wherein the sequence-based clustering algorithm is CD-HIT.
51. The method of any one of embodiments 38 to 50, further comprising a step of generating the initial set of CRISPR-Cas protein sequences.
52. The method of embodiment 51, wherein the step of generating the initial set of CRISPR-Cas proteins comprises identifying CRISPR-Cas loci from a set of bacterial and/or archaeal genomes.
53. The method of embodiment 52, wherein the step of generating the initial set of CRISPR-Cas proteins comprises identifying CRISPR-Cas loci using CRISPRCasTyper.
54. The method of any one of embodiments 1 to 53, wherein the viral genomes comprise phagic genomes.
55. The method of embodiment 54, wherein the viral genomes comprise phagic genomes of the human microbiome.
56. The method of any one of embodiments 1 to 55, wherein the set of viral genomes comprises at least 100,000 viral genomes.
57. The method of embodiment 56, wherein the set of viral genomes comprises at least 200,000 viral genomes.
58. The method of embodiment 56, wherein the set of viral genomes comprises at least 300,000 viral genomes.
59. The method of any one of embodiments 1 to 58, wherein the set of viral genomes comprises up to 1 million viral genomes.
60. The method of any one of embodiments 1 to 58, wherein the set of viral genomes comprises up to 750,000 viral genomes.
61. The method of any one of embodiments 1 to 58, wherein the set of viral genomes comprises up to 500,000 viral genomes.
62. The method of any one of embodiments 1 to 58, wherein the set of viral genomes comprises up to 400,000 viral genomes.
63. The method of any one of embodiments 1 to 62, which comprises identifying a sequence in the set of viral genomes as a putative protospacer sequence if a spacer aligns to the sequence with no more than four nucleotide mismatches or gaps.
64. The method of embodiment 63, which comprises identifying a sequence in the set of viral genomes as a putative protospacer sequence if a spacer aligns to the sequence with no more than three nucleotide mismatches or gaps.
65. The method of embodiment 63, which comprises identifying a sequence in the set of viral genomes as a putative protospacer sequence if a spacer aligns to the sequence with no more than two nucleotide mismatches or gaps.
66. The method of embodiment 63, which comprises identifying a sequence in the set of viral genomes as a putative protospacer sequence if a spacer aligns to the sequence with no more than one nucleotide mismatch or gap.
67. The method of embodiment 63, which comprises identifying a sequence in the set of viral genomes as a putative protospacer sequence if a spacer aligns to the sequence with no nucleotide mismatches or gaps.
68. The method of any one of embodiments 1 to 67, wherein aligning the protospacer sequences and their flanking upstream and downstream viral genome sequences is performed using MUSCLE.
69. The method of any one of embodiments 1 to 67, wherein aligning the protospacer sequences and their flanking upstream and downstream viral genome sequences is performed using MAFFT.
70. The method of any one of embodiments 1 to 69, wherein the flanking upstream and downstream sequences comprise up to 40 nucleotides upstream and up to 40 nucleotides downstream of the putative protospacer sequences.
71. The method of embodiment 70, wherein the flanking upstream and downstream sequences comprise up to 30 nucleotides upstream and up to 30 nucleotides downstream of the putative protospacer sequences.
72. The method of embodiment 70, wherein the flanking upstream and downstream sequences comprise 30 nucleotides upstream and 30 nucleotides downstream of the putative protospacer sequences.
73. The method of embodiment 70, wherein the flanking upstream and downstream sequences comprise up to 20 nucleotides upstream and up to 20 nucleotides downstream of the putative protospacer sequences.
74. The method of embodiment 70, wherein the flanking upstream and downstream sequences comprise up to 10 nucleotides upstream and up to 10 nucleotides downstream of the putative protospacer sequences.
75. The method of any one of embodiments 1 to 74, wherein predicting a PAM sequence recognized by one or more CRISPR-Cas proteins in the cluster from the sets of aligned putative protospacer and flanking sequences comprises:
   a) generating a consensus sequence from each set of aligned putative protospacer and flanking sequences to generate a set of consensus sequences, each consensus sequence comprising an upstream region and a downstream region;
   b) calculating nucleotide frequencies at each nucleotide position in the set of consensus sequences for the upstream and downstream regions; and
   c) identifying one or more conserved nucleotides in the upstream or downstream regions in the set of consensus sequences, wherein the presence of one or more conserved nucleotides indicates that the PAM sequence is positioned in the upstream or downstream consensus sequences.
76. The method of embodiment 75, wherein the consensus sequences comprise the most frequent base at each position in a set of aligned putative protospacer and flanking sequences, discarding positions having >50% gaps.
77. The method of embodiment 75 or embodiment 76, further comprising generating a sequence logo from the nucleotide frequencies.
78. The method of embodiment 77, wherein the one or more conserved nucleotides are nucleotides at positions in the sequence logo having a bit level of at least 1.
79. The method of embodiment 77 or embodiment 78, wherein the one or more conserved nucleotides are nucleotides at positions in the sequence logo having a bit level that is larger than the median bit level in the sequence logo plus 1.5 times the interquartile range of bit levels in the sequence logo.
80. The method of any one of embodiments 77 to 79, further comprising generating, in a computerized system, a report with the sequence logo.
81. The method of any one of embodiments 1 to 80, further comprising generating, in a computerized system, a report with the predicted PAM.
82. The method of any one of embodiments 1 to 81, further comprising validating the predicted PAM in vitro.
83. The method of any one of embodiments 1 to 82, wherein the CRISPR-Cas proteins comprise Type II Cas proteins.
84. The method of any one of embodiments 1 to 83, wherein the CRISPR-Cas proteins comprise Type II-A Cas proteins.
85. The method of any one of embodiments 1 to 84, wherein the CRISPR-Cas proteins comprise Type II-B Cas proteins.
86. The method of any one of embodiments 1 to 85, wherein the CRISPR-Cas proteins comprise Type II-C Cas proteins.
87. The method of any one of embodiments 1 to 86, wherein the CRISPR-Cas proteins comprise Cas9 proteins.
88. The method of any one of embodiments 1 to 87, wherein the CRISPR-Cas proteins comprise Type V Cas proteins.
89. The method of any one of embodiments 1 to 88, wherein the CRISPR-Cas proteins comprise Type V-A, Type V-B, Type V-C, Type V-D, Type V-E, Type V-F, Type V-G, Type V-H, Type V-I, Type V-J, or Type V-K Cas proteins.
90. The method of any one of embodiments 1 to 89, wherein the CRISPR-Cas proteins comprise Cas12a proteins.
91. The method of any one of embodiments 1 to 90, wherein the CRISPR-Cas proteins comprise Type VI Cas proteins.
92. The method of any one of embodiments 1 to 91, wherein the CRISPR-Cas proteins comprise Cas13 proteins.
93. The method of any one of embodiments 1 to 92, comprising performing steps (a)-(c) for more than one CRISPR-Cas protein cluster.
94. The method of embodiment 93, comprising performing steps (a)-(c) for up to 100,000 CRISPR-Cas protein clusters.
95. The method of embodiment 93 or embodiment 94, which comprises discarding clusters having fewer than 10 mapped spacers.
96. The method of any one of embodiments 93 to 95, further comprising clustering the CRISPR-Cas protein sequences by their predicted PAM sequences to generate PAM clusters.
97. The method of embodiment 96, further comprising performing hierarchical clustering of one or more PAM clusters.
98. The method of any one of embodiments 1 to 97, wherein the method is repeated using:
   a) a second CRISPR-Cas protein cluster;
   b) a second set of viral genomes; or
   c) a second CRISPR-Cas protein cluster and a second set of viral genomes.
99. The method of embodiment 98, which comprises repeating the method periodically.
100. The method of any one of embodiments 98 to 99, depending directly or indirectly from embodiment 38, wherein the method is repeated using a second initial set of CRISPR-Cas protein sequences.
101. A system configured to predict a PAM sequence according to the method of any one of embodiments 1 to 100.
102. The system of embodiment 101, which comprises one or more processors coupled to a memory storing one or more computer readable instructions for execution by the one or more processors.
103. A tangible, non-transitory computer-readable media comprising instructions executable by a processor for executing a method according to any one of embodiments 1 to 100.
104. A tangible, non-transitory computer-readable media comprising a library of CRISPR-Cas protein sequences with predicted PAM sequences obtained or obtainable by the method of any one of embodiments 1 to 100.
105. A method of identifying a PAM sequence recognized by a CRISPR-Cas protein, comprising:
   a) predicting a PAM sequence recognized by the CRISPR-Cas protein according to the method of any one of embodiments 1 to 100; and
   b) validating the predicted PAM in vitro, thereby identifying the PAM sequence.
106. A method for predicting a PAM sequence recognized by a CRISPR-Cas protein of interest, comprising:
   a) identifying, in a library of CRISPR-Cas proteins with predicted PAM sequences obtained or obtainable by the method of any one of embodiments 1 to 100, the CRISPR-Cas protein having the highest amino acid sequence identity with the amino acid sequence of the CRISPR-Cas protein of interest; and
   b) predicting that the PAM sequence of the CRISPR-Cas protein of interest is the same as the predicted PAM sequence of the CRISPR-Cas protein in the library.
107. The method of embodiment 106, which is computer implemented.
108. The method of embodiment 106 or embodiment 107, further comprising validating the predicted PAM of the CRISPR-Cas protein of interest in vitro.
109. A method of selecting a CRISPR-Cas protein for editing a genomic sequence of interest, comprising:
   a) identifying, in a library of CRISPR-Cas protein sequences with predicted PAM sequences obtained or obtainable by the method of any one of embodiments 1 to 100, a predicted PAM sequence in the library whose sequence is present in the genomic sequence of interest; and
   b) selecting a CRISPR-Cas protein from the library whose predicted PAM sequence is present in the genomic sequence of interest.
110. The method of embodiment 109, wherein identifying a predicted PAM sequence in the library whose sequence is present in the genomic sequence of interest comprises aligning one or more predicted PAM sequences in the CRISPR-Cas protein library to the genomic sequence of interest.
111. The method of embodiment 109, wherein identifying a predicted PAM sequence in the library whose sequence is present in the genomic sequence of interest comprises aligning each predicted PAM sequence in the CRISPR-Cas protein library to the genomic sequence of interest.
112. A method of selecting a CRISPR-Cas protein for editing a genomic sequence of interest comprising a PAM sequence of interest, comprising selecting a CRISPR-Cas protein from a library of CRISPR-Cas protein sequences with predicted PAM sequences obtained or obtainable by the method of any one of embodiments 1 to 100, wherein the predicted PAM sequence of the selected CRISPR-Cas protein corresponds to the PAM sequence of interest.
113. The method of any one of embodiments 109 to 112, wherein the step of selecting the CRISPR-Cas protein is computer implemented.
114. The method of any one of embodiments 109 to embodiment 113, further comprising evaluating the ability of the CRISPR-Cas protein to edit the genomic sequence.
115. The method of embodiment 114, wherein evaluating the ability of the selected CRISPR-Cas protein to edit the genomic sequence comprises an in vitro gene editing assay.
116. A method of selecting a CRISPR-Cas protein for editing a genomic sequence of interest, comprising:
   a) identifying, in a library of CRISPR-Cas protein sequences with predicted PAM sequences obtained or obtainable by the method of any one of embodiments 1 to 100, PAM sequences whose sequences are present in the genomic sequence of interest; and
   b) identifying one or more CRISPR-Cas proteins from the library whose predicted PAM sequence(s) are present in the genomic sequence of interest;
   c) evaluating the ability of the one or more CRISPR-Cas proteins to edit the genomic sequence; and
   d) selecting a CRISPR-Cas protein from the one or more CRISPR-Cas proteins following the evaluation, thereby selecting a CRISPR-Cas protein for editing the genomic sequence.
117. A method of selecting a CRISPR-Cas protein for editing a genomic sequence comprising a PAM sequence of interest, comprising:
   a) identifying one or more CRISPR-Cas proteins from a library of CRISPR-Cas protein sequences with predicted PAM sequences obtained or obtainable by the method of any one of embodiments 1 to 100, wherein the predicted PAM sequences of the one or more CRISPR-Cas proteins correspond to the PAM sequence of interest; and
   b) evaluating the ability of the one or more CRISPR-Cas proteins to edit the genomic sequence; and
   c) selecting a CRISPR-Cas protein from the one or more CRISPR-Cas proteins following the evaluation, thereby selecting a CRISPR-Cas protein for editing the genomic sequence comprising the PAM sequence of interest.
118. The method of embodiment 116 or embodiment 117, wherein the step of identifying the one or more CRISPR-Cas proteins is computer implemented.
119. The method any one of embodiments 116 to 118, wherein evaluating the ability of the one or more CRISPR-Cas proteins to edit the genomic sequence comprises an in vitro gene editing assay.
120. The method of any one of embodiments 110 to 117, when depending directly or indirectly from embodiment 110 or embodiment 117, wherein the PAM of interest is a PAM sequence created by a pathogenic mutation.
121. The method of embodiment 120, wherein the pathogenic mutation is an autosomal dominant mutation.
122. The method of embodiment 120, wherein the autosomal dominant mutation is in a RHO gene.
123. The method of embodiment 122, wherein the autosomal dominant mutation corresponds to the P23H mutation.
124. The method of any one of embodiments 109 to 123, further comprising designing a guide RNA (gRNA) molecule for editing the genomic sequence with the selected CRISPR-Cas protein.
125. A method of designing a guide RNA (gRNA) molecule, comprising designing a guide RNA (gRNA) molecule for a CRISPR-Cas protein identified according to the method of any one of embodiments 109 to 123.
126. The method of embodiment 124 or embodiment 125, wherein the design of the gRNA is computer implemented.
127. The method of any one of embodiments 124 to 126, which comprises identifying a tracrRNA sequence of the selected CRISPR-Cas protein, optionally wherein identifying a tracrRNA sequence of the selected CRISPR-Cas protein is computer-implemented.
128. The method of any one of embodiments 124 to 127, wherein the gRNA molecule comprises a crRNA comprising a targeting sequence and a tracrRNA, optionally wherein the gRNA is a sgRNA molecule.
129. The method of any one of embodiments 124 to 128, further comprising synthesizing the gRNA molecule or a nucleic acid encoding the gRNA molecule.
130. A method for editing a genomic sequence, comprising contacting a cell comprising the genomic sequence with a system comprising a CRISPR-Cas protein selected according to the method of any one of embodiments 109 to 124 and a gRNA for editing the genomic sequence, optionally wherein the gRNA is a gRNA designed according to any one of embodiments 125 to 128.
131. The method of embodiment 130, which comprises contacting the cell with one or more nucleic acids encoding the CRISPR-Cas protein and the gRNA.
132. The method of embodiment 131, wherein the CRISPR-Cas protein and the gRNA are encoded by the same nucleic acid.
133. The method of embodiment 131, wherein the CRISPR-Cas protein and the gRNA are encoded by different nucleic acids.
134. The method of any one of embodiments 131 to 133, wherein the nucleic acid(s) is/are AAV vector genome(s).
135. The method of embodiment 134, which comprises contacting the cell with one or more AAV particles comprising the vector genome(s).
136. The method of embodiment 130, which comprises contacting the cell with a ribonucleoprotein complex comprising the CRISPR-Cas protein and the gRNA.
137. A method of selecting a genomic sequence having a pathogenic mutation for editing with a CRISPR-Cas protein, comprising:
   a) identifying, in a library of CRISPR-Cas protein sequences with predicted PAM sequences obtained or obtainable by the method of any one of embodiments 1 to 100, a PAM sequence associated with a pathogenic mutation; and
   b) selecting a genomic sequence having the PAM sequence associated with the pathogenic mutation for editing with a CRISPR-Cas protein.
138. The method of embodiment 137, further comprising selecting one or more CRISPR-Cas proteins from the library whose predicted PAM corresponds to the PAM sequence associated with the pathogenic mutation.
139. The method of embodiment 138, further comprising designing a gRNA for editing the genomic sequence having the pathogenic mutation with one or more of the selected CRISPR-Cas proteins, optionally wherein the gRNA is designed according to the method of any one of embodiments 124 to 128 and/or synthesized according to the method of embodiment 129.
140. The method of embodiment 138 or embodiment 139, further comprising editing the genomic sequence having the pathogenic mutation with one or more of the selected CRISPR-Cas proteins, optionally wherein the editing comprises contacting a cell comprising the genomic sequence having the pathogenic mutation with a CRISPR-Cas protein and a gRNA according to the method of any one of embodiments 130 to 136.
141. The method of any one of embodiments 138 to 140, further comprising evaluating the ability of the one or more selected CRISPR-Cas proteins to edit the genomic sequence having the pathogenic mutation.
142. The method of embodiment 141, wherein evaluating the ability of the one or more CRISPR-Cas proteins to edit the genomic sequence comprises an in vitro gene editing assay.

### 9. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there are any inconsistencies between the teachings of one or more of the references incorporated herein and the present disclosure, the teachings of the present specification are intended.

What is claimed is:

1. A method for predicting a protospacer adjacent motif (PAM) sequence recognized by one or more CRISPR-Cas proteins in a CRISPR-Cas protein cluster, comprising:
a) mapping spacer sequences of one or more CRISPR arrays corresponding to one or more CRISPR-Cas proteins of the CRISPR-Cas protein cluster to a set of viral genome sequences to identify putative protospacer sequences in the viral genomes and their flanking upstream and downstream sequences in the viral genomes, wherein:
i) mapping the spacer sequences to the viral genomes comprises aligning the spacer sequences to the viral genome sequences; and
ii) the CRISPR-Cas protein cluster comprises a set of CRISPR-Cas protein sequences clustered by sequence identity;
b) for each spacer sequence mapped to one or more viral genome sequences, aligning the putative protospacer sequences and their flanking upstream and downstream viral genome sequences to generate a set of aligned putative protospacer and flanking sequences for each mapped spacer sequence; and
c) predicting a PAM sequence recognized by one or more CRISPR-Cas proteins in the cluster from the sets of aligned putative protospacer and flanking sequences.

2. The method of claim 1, which is computer implemented.

3. A computer-implemented method for predicting a protospacer adjacent motif (PAM) sequence recognized by one or more CRISPR-Cas proteins in a CRISPR-Cas protein cluster, the method comprising executing, in a computer system having one or more processors coupled to a memory storing one or more computer readable instructions for execution by the one or more processors, the one or more computer readable instructions comprising instructions for:
a) mapping spacer sequences of one or more CRISPR arrays corresponding to one or more CRISPR-Cas proteins of the CRISPR-Cas protein cluster to a set of viral genome sequences to identify putative protospacer sequences in the viral genomes and their flanking upstream and downstream sequences in the viral genomes, wherein:
i) mapping the spacer sequences to the viral genomes comprises aligning the spacer sequences to the viral genome sequences; and
ii) the CRISPR-Cas protein cluster comprises a set of CRISPR-Cas protein sequences clustered by sequence identity;
b) for each spacer sequence mapped to putative protospacer sequences, aligning the protospacer sequences and their flanking upstream and downstream viral genome sequences to generate a set of aligned putative protospacer and flanking sequences for each mapped spacer sequence; and
c) predicting a PAM sequence recognized by one or more CRISPR-Cas proteins in the cluster from the sets of aligned putative protospacer and flanking sequences.

4. The method of any one of claims 1 to 3, wherein the CRISPR-Cas protein cluster comprises at least 1, at least 10, at least 100, at least 1000, at least 2000, at least 5000, or at least 10,000 CRISPR-Cas protein sequences.

5. The method of any one of claims 1 to 4, wherein the CRISPR-Cas protein sequences in the cluster are at least 150, at least 200, at least 500, at least 700, at least 900, at least 950, at least 1000, at least 1100, at least 1200, or at least 1300 amino acids in length.

6. The method of any one of claims 1 to 5, wherein the CRISPR-Cas protein sequences in the cluster are less than 2200, less than 2100, less than 2000, less than 1800, less than 1600, less than 1500, less than 1400, less than 1300, less than 1200, or less than 1100 amino acids in length.

7. The method of any one of claims 1 to 6, wherein each CRISPR-Cas protein sequence in the cluster has at least 95%, at least 96%, at least 97%, at least 98%, at least 99%, or 100% sequence identity to a centroid sequence.

8. The method of any one of claims 1 to 7, further comprising a step of clustering an initial set of CRISPR-Cas protein sequences to generate the CRISPR-Cas protein cluster.

9. The method of claim 8, wherein the initial set of CRISPR-Cas protein sequences comprises at least 10,000, at least 50,000, at least 75,000, or at least 90,000 CRISPR-Cas protein sequences.

10. The method of claim 8 or claim 9, wherein the initial set of CRISPR-Cas protein sequences comprises up to 1 million, up to 750,000, up to 500,000, up to 200,000, or up to 100,000 CRISPR-Cas protein sequences.

11. The method of any one of claims 8 to 10, wherein the step of clustering the initial set of CRISPR-Cas protein sequences comprises clustering the initial set of CRISPR-Cas protein sequences by a sequence-based clustering algorithm.

12. The method of claim 11, wherein the sequence-based clustering algorithm is UCLUST or CD-HIT.

13. The method of any one of claims 8 to 12, further comprising a step of generating the initial set of CRISPR-Cas protein sequences.

14. The method of claim 13, wherein the step of generating the initial set of CRISPR-Cas proteins comprises identifying CRISPR-Cas loci from a set of bacterial and/or archaeal genomes.

15. The method of claim 14, wherein the step of generating the initial set of CRISPR-Cas proteins comprises identifying CRISPR-Cas loci using CRISPRCasTyper.

16. The method of any one of claims 1 to 15, wherein the viral genomes comprise phagic genomes.

17. The method of claim 16, wherein the viral genomes comprise phagic genomes of the human microbiome.

18. The method of any one of claims 1 to 17, wherein the set of viral genomes comprises at least 100,000, at least 200,000, or at least 300,000 viral genomes.

19. The method of any one of claims 1 to 18, wherein the set of viral genomes comprises up to 1 million, up to 750,000, up to 500,000, or up to 400,000 viral genomes.

20. The method of any one of claims 1 to 19, which comprises identifying a sequence in the set of viral genomes as a putative protospacer sequence if a spacer aligns to the sequence with no more than four nucleotide mismatches or gaps, no more than three nucleotide mismatches or gaps, no more than two nucleotide mismatches or gap, no more than one nucleotide mismatch or gap, or no nucleotide mismatches or gaps.

21. The method of any one of claims 1 to 20, wherein aligning the protospacer sequences and their flanking upstream and downstream viral genome sequences is performed using MUSCLE or MAFFT.

22. The method of any one of claims 1 to 21, wherein the flanking upstream and downstream sequences comprise (i) up to 40 nucleotides upstream and up to 40 nucleotides downstream of the putative protospacer sequences, (ii) up to 30 nucleotides upstream and up to 30 nucleotides downstream of the putative protospacer sequences, (iii) 30 nucleotides upstream and 30 nucleotides downstream of the putative protospacer sequences, (iv) up to 20 nucleotides upstream and up to 20 nucleotides downstream of the putative protospacer sequences, or (v) up to 10 nucleotides upstream and up to 10 nucleotides downstream of the putative protospacer sequences.

23. The method of any one of claims 1 to 22, wherein predicting a PAM sequence recognized by one or more CRISPR-Cas proteins in the cluster from the sets of aligned putative protospacer and flanking sequences comprises:
a) generating a consensus sequence from each set of aligned putative protospacer and flanking sequences to generate a set of consensus sequences, each consensus sequence comprising an upstream region and a downstream region;
b) calculating nucleotide frequencies at each nucleotide position in the set of consensus sequences for the upstream and downstream regions; and
c) identifying one or more conserved nucleotides in the upstream or downstream regions in the set of consensus sequences, wherein the presence of one or more conserved nucleotides indicates that the PAM sequence is positioned in the upstream or downstream consensus sequences.

24. The method of claim 23, wherein the consensus sequences comprise the most frequent base at each position in a set of aligned putative protospacer and flanking sequences, discarding positions having >50% gaps.

25. The method of claim 23 or claim 24, further comprising generating a sequence logo from the nucleotide frequencies.

26. The method of claim 25, wherein the one or more conserved nucleotides are nucleotides at positions in the sequence logo having a bit level of at least 1.

27. The method of claim 25 or claim 26, wherein the one or more conserved nucleotides are nucleotides at positions in the sequence logo having a bit level that is larger than the median bit level in the sequence logo plus 1.5 times the interquartile range of bit levels in the sequence logo.

28. The method of any one of claims 25 to 27, further comprising generating, in a computerized system, a report with the sequence logo.

29. The method of any one of claims 1 to 28, further comprising generating, in a computerized system, a report with the predicted PAM.

30. The method of any one of claims 1 to 29, further comprising validating the predicted PAM in vitro.

31. The method of any one of claims 1 to 30, wherein the CRISPR-Cas proteins comprise Type II Cas proteins, optionally wherein the CRISPR-Cas proteins comprise Type II-A Cas proteins, Type II-B Cas proteins, or Type II-C Cas proteins.

32. The method of any one of claims 1 to 31, wherein the CRISPR-Cas proteins comprise Cas9 proteins.

33. The method of any one of claims 1 to 32, wherein the CRISPR-Cas proteins comprise Type V Cas proteins, optionally wherein the CRISPR-Cas proteins comprise Type V-A, Type V-B, Type V-C, Type V-D, Type V-E, Type V-F, Type V-G, Type V-H, Type V-I, Type V-J, or Type V-K Cas proteins, optionally wherein the CRISPR-Cas proteins comprise Cas12a proteins.

34. The method of any one of claims 1 to 33, wherein the CRISPR-Cas proteins comprise Type VI Cas proteins, optionally wherein the CRISPR-Cas proteins comprise Cas13 proteins.

35. The method of any one of claims 1 to 34, comprising performing steps (a)-(c) for more than one CRISPR-Cas protein cluster and optionally performing steps (a)-(c) for up to 100,000 CRISPR-Cas protein clusters.

36. The method of claim 35, which comprises discarding clusters having fewer than 10 mapped spacers.

37. The method of claim 35 or claim 36, further comprising clustering the CRISPR-Cas protein sequences by their predicted PAM sequences to generate PAM clusters, optionally further comprising performing hierarchical clustering of one or more PAM clusters.

38. The method of any one of claims 1 to 37, wherein the method is repeated using:
a) a second CRISPR-Cas protein cluster;
b) a second set of viral genomes; or
c) a second CRISPR-Cas protein cluster and a second set of viral genomes.

39. The method of claim 38, which comprises repeating the method periodically.

40. The method of claim 38 or claim 39, depending directly or indirectly from claim 8, wherein the method is repeated using a second initial set of CRISPR-Cas protein sequences.

41. A system configured to predict a PAM sequence according to the method of any one of claims 1 to 40.

42. The system of claim 41, which comprises one or more processors coupled to a memory storing one or more computer readable instructions for execution by the one or more processors.

43. A tangible, non-transitory computer-readable media comprising instructions executable by a processor for executing a method according to any one of claims 1 to 40.

44. A tangible, non-transitory computer-readable media comprising a library of CRISPR-Cas protein sequences with predicted PAM sequences obtained or obtainable by the method of any one of claims 1 to 40.

45. A method of identifying a PAM sequence recognized by a CRISPR-Cas protein, comprising:
a) predicting a PAM sequence recognized by the CRISPR-Cas protein according to the method of any one of claims 1 to 40; and
b) validating the predicted PAM in vitro, thereby identifying the PAM sequence.

46. A method for predicting a PAM sequence recognized by a CRISPR-Cas protein of interest, comprising:
a) identifying, in a library of CRISPR-Cas proteins with predicted PAM sequences obtained or obtainable by the method of any one of claims 1 to 40, the CRISPR-Cas protein having the highest amino acid sequence identity with the amino acid sequence of the CRISPR-Cas protein of interest; and
b) predicting that the PAM sequence of the CRISPR-Cas protein of interest is the same as the predicted PAM sequence of the CRISPR-Cas protein in the library;
optionally wherein the method is computer implemented.

47. The method of claim 46, further comprising validating the predicted PAM of the CRISPR-Cas protein of interest in vitro.

48. A method of selecting a CRISPR-Cas protein for editing a genomic sequence of interest, comprising:
a) identifying, in a library of CRISPR-Cas protein sequences with predicted PAM sequences obtained or obtainable by the method of any one of claims 1 to 40, a predicted PAM sequence in the library whose sequence is present in the genomic sequence of interest; and
b) selecting a CRISPR-Cas protein from the library whose predicted PAM sequence is present in the genomic sequence of interest.

49. The method of claim 48, wherein identifying a predicted PAM sequence in the library whose sequence is present in the genomic sequence of interest comprises aligning one or more predicted PAM sequences in the CRISPR-Cas protein library to the genomic sequence of interest.

50. The method of claim 48, wherein identifying a predicted PAM sequence in the library whose sequence is present in the genomic sequence of interest comprises aligning each predicted PAM sequence in the CRISPR-Cas protein library to the genomic sequence of interest.

51. A method of selecting a CRISPR-Cas protein for editing a genomic sequence of interest comprising a PAM sequence of interest, comprising selecting a CRISPR-Cas protein from a library of CRISPR-Cas protein sequences with predicted PAM sequences obtained or obtainable by the method of any one of claims 1 to 40, wherein the predicted PAM sequence of the selected CRISPR-Cas protein corresponds to the PAM sequence of interest.

52. The method of any one of claims 48 to 51, wherein the step of selecting the CRISPR-Cas protein is computer implemented.

53. The method of any one of claim 48 to claim 52, further comprising evaluating the ability of the CRISPR-Cas protein to edit the genomic sequence.

54. The method of claim 53, wherein evaluating the ability of the selected CRISPR-Cas protein to edit the genomic sequence comprises an in vitro gene editing assay.

55. A method of selecting a CRISPR-Cas protein for editing a genomic sequence of interest, comprising:
a) identifying, in a library of CRISPR-Cas protein sequences with predicted PAM sequences obtained or obtainable by the method of any one of claims 1 to 40, PAM sequences whose sequences are present in the genomic sequence of interest; and
b) identifying one or more CRISPR-Cas proteins from the library whose predicted PAM sequence(s) are present in the genomic sequence of interest;
c) evaluating the ability of the one or more CRISPR-Cas proteins to edit the genomic sequence; and
d) selecting a CRISPR-Cas protein from the one or more CRISPR-Cas proteins following the evaluation, thereby selecting a CRISPR-Cas protein for editing the genomic sequence.

56. A method of selecting a CRISPR-Cas protein for editing a genomic sequence comprising a PAM sequence of interest, comprising:
a) identifying one or more CRISPR-Cas proteins from a library of CRISPR-Cas protein sequences with predicted PAM sequences obtained or obtainable by the method of any one of claims 1 to 40, wherein the predicted PAM sequences of the one or more CRISPR-Cas proteins correspond to the PAM sequence of interest; and
b) evaluating the ability of the one or more CRISPR-Cas proteins to edit the genomic sequence; and
c) selecting a CRISPR-Cas protein from the one or more CRISPR-Cas proteins following the evaluation, thereby selecting a CRISPR-Cas protein for editing the genomic sequence comprising the PAM sequence of interest.

57. The method of claim 55 or claim 56, wherein the step of identifying the one or more CRISPR-Cas proteins is computer implemented.

58. The method any one of claims 55 to 57, wherein evaluating the ability of the one or more CRISPR-Cas proteins to edit the genomic sequence comprises an in vitro gene editing assay.

59. The method of any one of claims 49 to 56, when depending directly or indirectly from claim 49 or claim 56, wherein the PAM of interest is a PAM sequence created by a pathogenic mutation, optionally wherein the pathogenic mutation is an autosomal dominant mutation, optionally wherein the autosomal dominant mutation is in a RHO gene, optionally wherein the autosomal dominant mutation in a RHO gene corresponds to the P23H mutation.

60. The method of any one of claims 48 to 59, further comprising designing a guide RNA (gRNA) molecule for editing the genomic sequence with the selected CRISPR-Cas protein.

61. A method of designing a guide RNA (gRNA) molecule, comprising designing a guide RNA (gRNA) molecule for a CRISPR-Cas protein identified according to the method of any one of claims 48 to 59.

62. The method of claim 60 or claim 61, wherein the design of the gRNA is computer implemented.

63. The method of any one of claims 60 to 62, which comprises identifying a tracrRNA sequence of the selected CRISPR-Cas protein, optionally wherein identifying a tracrRNA sequence of the selected CRISPR-Cas protein is computer-implemented.

64. The method of any one of claims 60 to 63, wherein the gRNA molecule comprises a crRNA comprising a targeting sequence and a tracrRNA, optionally wherein the gRNA is a sgRNA molecule.

65. The method of any one of claims 60 to 64, further comprising synthesizing the gRNA molecule or a nucleic acid encoding the gRNA molecule.

66. A method for editing a genomic sequence, comprising contacting a cell comprising the genomic sequence with a system comprising a CRISPR-Cas protein selected according to the method of any one of claims 48 to 60 and a gRNA for editing the genomic sequence, optionally wherein the gRNA is a gRNA designed according to any one of claims 61 to 64.

67. A method of selecting a genomic sequence having a pathogenic mutation for editing with a CRISPR-Cas protein, comprising:
a) identifying, in a library of CRISPR-Cas protein sequences with predicted PAM sequences obtained or obtainable by the method of any one of claims 1 to 40, a PAM sequence associated with a pathogenic mutation; and
b) selecting a genomic sequence having the PAM sequence associated with the pathogenic mutation for editing with a CRISPR-Cas protein.

68. The method of claim 67, further comprising selecting one or more CRISPR-Cas proteins from the library whose predicted PAM corresponds to the PAM sequence associated with the pathogenic mutation.

69. The method of claim 68, further comprising designing a gRNA for editing the genomic sequence having the pathogenic mutation with one or more of the selected CRISPR-Cas proteins, optionally wherein the gRNA is designed according to the method of any one of claims 60 to 64 and/or synthesized according to the method of claim 65.

70. The method of claim 68 or claim 69, further comprising editing the genomic sequence having the pathogenic mutation with one or more of the selected CRISPR-Cas proteins, optionally wherein the editing comprises contacting a cell comprising the genomic sequence having the pathogenic mutation with a CRISPR-Cas protein and a gRNA according to the method of claim 66.

71. The method of any one of claims 68 to 70, further comprising evaluating the ability of the one or more selected CRISPR-Cas proteins to edit the genomic sequence having the pathogenic mutation.

72. The method of claim 71, wherein evaluating the ability of the one or more CRISPR-Cas proteins to edit the genomic sequence comprises an in vitro gene editing assay.
